(19) United States
(12) Patent Application Publication
(10) Pub. No.: US 2008/0312160 A1
(43) Pub. Date: Dec. 18, 2008

(54) METHOD OF TREATING ENTERITIS, INTESTINAL DAMAGE, AND DIARRHEA FROM C. DIFFICILE WITH AN A2A ADENOSINE RECEPTOR AGONIST

(76) Inventors: Richard L. Guerrant, Charlottesville, VA (US); Joel M. Linden, Charlottesville, VA (US); Cirle A. Warren, Waynesboro, VA (US); Gail W. Sullivan, Charlottesville, VA (US); Timothy L. Macdonald, Charlottesville, VA (US)

Correspondence Address: SCHWEGMAN, LUNDBERG & WOESSNER, P.A., P.O. BOX 2938, MINNEAPOLIS, MN, 55402, US

(21) Appl. No.: 12/100,071

(22) Filed: Apr. 9, 2008

Related U.S. Application Data
(60) Provisional application No. 60/922,532, filed on Apr. 9, 2007.
(60) Provisional application No. 60/912,080, filed on Apr. 16, 2007.

(51) Int. Cl. A61K 38/05; A61K 31/7076; A61P 31/04

(52) U.S. Cl. 514/19; 514/46

(57) ABSTRACT

A therapeutic method for treating intestinal damage, enteritis, diarrhea, or a combination thereof caused by a C. difficile is provided. The method includes administration to a patient in need thereof an effective amount of an A_{2A} adenosine receptor agonist, optionally in combination with an effective amount of a stable glutamine derivative, e.g., alanyl-glutamine.

### RELATED APPLICATIONS

This application claims priority from U.S. Provisional Application Ser. Nos. 60/922,532, filed Apr. 9, 2007, and 60/912,080, filed Apr. 16, 2007, which applications are herein incorporated by reference.

### FIELD OF THE INVENTION

The present invention relates to a method for treating C. difficile or C. difficile toxin A induced intestinal damage, enteritis, diarrhea, or a combination thereof with an A_{2A} adenosine receptor agonist, optionally in combination with a stable glutamine derivative (e.g., alanyl-glutamine).

### BACKGROUND OF THE INVENTION

Clostridium difficile (C. difficile) is the most common cause of nosocomial bacterial diarrhea and accounts for 10-20% of the cases of antibiotic-associated diarrhea. C. difficile infection can result in asymptomatic carriage, mild diarrhea, or fulminant pseudomembranous colitis. This anaerobic bacterium causes intestinal damage through the actions of two large exotoxins, toxin A and toxin B. A third toxin, designated CDT with actin-specific ADP-ribosyltransferase activity was described from C. difficile strain CD196 in 1988. Strains carrying CDT genes may be associated with the severity of C. difficile disease. Purified toxin A (TxA) causes intestinal secretion, destruction of the intestinal epithelium and hemorrhagic colitis when introduced in vivo to the intestinal lumen. The mechanism of TxA-induced enteritis involves toxin binding to enterocyte receptors, leading to activation of sensory and enteric nerves that results in enhanced intestinal secretion and motility, degranulation of mast cells, and infiltration of the mucosa by neutrophils. In addition to its proinflammatory and prosecretory activities, TxA induces apoptosis and nonapoptotic cell death in human and murine cells, which could contribute to intestinal mucosal disruption.

C. difficile infections are common worldwide and there is a need for new methods for treating the numerous conditions caused by C. difficile and C. difficile toxin A.

### SUMMARY OF THE INVENTION

In an aspect, the present invention provides a novel method of treating C. difficile or C. difficile toxin A induced intestinal damage, enteritis, diarrhea, or a combination thereof with an A_{2A} adenosine receptor agonist, optionally in combination with a stable glutamine derivative (e.g., alanyl-glutamine).

In another aspect, the present invention provides a novel use of an A_{2A} adenosine receptor agonist, optionally in combination with a stable glutamine derivative, for medical therapy.

In another aspect, the present invention provides a novel use of an A_{2A} adenosine receptor agonist, optionally in combination with a stable glutamine derivative, for the manufacture of a medicament for treating a disease described herein.

Unexpectedly, it was found that administration of an A_{2A} adenosine receptor agonist alone and also in combination with alanyl-glutamine reduced the damaging effects of C. Difficile and/or C. Difficile Toxin A.

### BRIEF DESCRIPTION OF THE DRAWINGS

FIGS. 1A and B show the effect of C. Difficile toxin A (TxA) on weight (A) and secretion volume (B) of murine ileal loops.

FIGS. 2A and B show the effect of ATL 313 on weight (A) and secretion volume (B) on murine ileal loops injected with C. Difficile TxA.

FIG. 3 shows the effect of ATL 313 on C. Difficile TxA-induced TNF-α production.

FIGS. 4A and B show the effect of ATL 313 on C. Difficile TxA-induced adenosine deaminase (ADA) production.

FIGS. 5 and 6 show the effect of different doses of A_{2A} agonists, JMR 193 and ATL 146e, on C. difficile toxin A-induced inflammation in rabbit ileal loops. The Y-axis is the histology score graded from 0 (none) to 4 (worst) based on the degree of mucosal disruption, increase in cellularity, and intensity of vascular congestion.

FIGS. 7 and 8 show the effect of the A_{2A} agonists on toxin A-induced secretion. The Y-axis is the volume to length ratio in mL/cm (amount of intestinal fluid divided by the length of the intestinal loop).

FIG. 9 shows the effect of A_{2A} agonist ATL 313 (100 nM) alone versus ATL 313 plus alanyl-glutamine (25 mM) on Toxin A-induced secretion. The Y-axis is the volume to length ratio as described above.

FIG. 10 shows the alanyl-glutamine (AQ) dose response inhibition of Toxin A-induced secretion.

FIG. 11 shows ATL 370 inhibition of Toxin A-induced secretion alone or in dose response with alanyl-glutamine (AQ).

### DETAILED DESCRIPTION OF THE INVENTION

In an embodiment, the present invention provides a novel method of treating C. difficile or C. difficile toxin A induced intestinal damage, enteritis, diarrhea, or a combination thereof, comprising administering an effective amount of an A_{2A} adenosine receptor agonist.

In another embodiment, the present invention provides a novel method of treating C. difficile or C. difficile toxin A induced intestinal damage, enteritis, diarrhea, or a combination thereof, comprising administering an effective amount of an A_{2A} adenosine receptor agonist, optionally in combination with an effective amount of a stable glutamine derivative.

In another embodiment, the stable glutamine derivative is an oligopeptide having from 2 to 5 amino acid units and containing therein the amino acid sequence alanine-glutamine. Such derivatives are described in U.S. Pat. No. 5,561,111, which is incorporated herein by reference.

In another embodiment, the stable glutamine derivative is selected from alanyl-glutamine and alanyl-glutaminyl glutamine.

In another embodiment, the stable glutamine derivative is alanyl-glutamine.

In another embodiment, the present invention provides a novel use of an A_{2A} adenosine receptor agonist for medical therapy.

In another embodiment, the present invention provides a novel use of a combination of an A_{2A} adenosine receptor agonist and a stable glutamine derivative for medical therapy.

In another embodiment, the present invention provides a novel use of an A_{2A} adenosine receptor agonist for the manufacture of a medicament for treating induced intestinal damage, enteritis, diarrhea, or a combination thereof.

In another embodiment, the present invention provides a novel use of a combination of an A_{2A} adenosine receptor agonist and a stable glutamine derivative for the manufacture of a medicament for treating induced intestinal damage, enteritis, diarrhea, or a combination thereof.

The combination of A_{2A} adenosine receptor agonist and a stable glutamine derivative can be a synergistic combination. Synergy, as described, for example, by Chou and Talalay, Adv. Enzyme Regul. 1984, 22:27-55, occurs when the effect of the compounds administered in combination is greater than the additive effect of the compounds when administered alone as a single agent. In general, a synergistic effect is most clearly demonstrated at sub-optimal concentrations of the compounds. Synergy can be in terms of lower cytotoxicity, increased effect, or some other beneficial effect of the combination compared with the individual components.

By “administering in combination” or “combination therapy” it is meant that the agents are administered concurrently to the mammal being treated. When administered in combination each agent can be administered at the same time (simultaneously) or sequentially in any order at different points in time (e.g., within minutes or hours). Thus, each agent may be administered separately, but sufficiently close in time so as to provide the desired therapeutic effect. For example, the administration of the A_{2A} adenosine receptor agonists can be within about 24 hours and within about 12 hours of the second agent. Each agent can be administered via the same or a different route (e.g., orally and parenterally).

In another embodiment, the invention provides a therapeutic method for treating a C. difficile infection with a combination of an A_{2A} adenosine receptor agonist and at least one antibiotic. This combination can further include a stable-glutamine derivative.

Examples of agonists of A_{2A} adenosine receptors that are expected to useful in the practice of the present invention include compounds having formula I or a stereoisomer or pharmaceutically acceptable salt thereof:

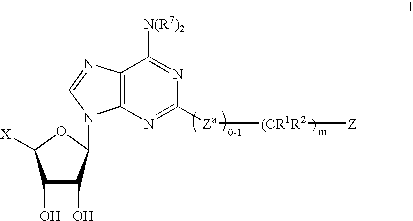

wherein

Z^{a} is C≡C, O, NH, or NHN═CR^{3a};

Z is CR³R⁴R⁵ or NR⁴R⁵;

each R¹ is independently hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S), —SSR^{a}, R^{a}S(═O)—, R^{a}S(═O)₂—, or —N═NR^{b};

each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-;

alternatively, R¹ and R² and the atom to which they are attached is C═O, C═S or C═NR^{d},

R⁴ and R⁵ are independently H or (C₁-C₈)alkyl;

alternatively, R⁴ and R⁵ together with the atom to which they are attached form a saturated, partially unsaturated, or aromatic ring that is mono-, bi- or polycyclic and has 3, 4, 5, 6, 7, 8, 9 or 10 ring atoms optionally having 1, 2, 3, or 4 heteroatoms selected from non-peroxide oxy (—O—), thio (—S—), sulfinyl (—SO—), sulfonyl (—S(O)₂—) or amine (—NR^{b}—) in the ring;

wherein R⁴ and R⁵ are independently substituted with 0-3 R⁶ groups or any ring comprising R⁴ and R⁵ is substituted with from 0 to 6 R⁶ groups;

each R⁶ is independently hydrogen, halo, —OR^{a}, —SR^{a}, (C₁-C₈)alkyl, cyano, nitro, trifluoromethyl, trifluoromethoxy, (C₁-C₈)cycloalkyl, (C₆-C₁₂)bicycloalkyl, heterocycle, heterocycle (C₁-C₈)alkylene-, aryl, aryl (C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups together with the atom or atoms to which they are attached can form a carbocyclic or heterocyclic ring comprising from 1-6 carbon atoms and 1, 2, 3, or 4 heteroatoms selected from non-peroxide oxy (—O—), thio (—S—), sulfinyl (—SO—), sulfonyl (—S(O)₂—) or amine (—NR^{b}—) in the ring;

R³ is hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, —NNR^{b}; or if the ring formed from CR⁴R⁵ is aryl or heteroaryl or partially unsaturated then R³ can be absent;

R^{3a} is hydrogen, (C₁-C₈)alkyl, or aryl;

each R⁷ is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;

X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c}C);

alternatively, X is an aromatic ring of the formula:

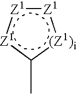

each Z¹ is independently non-peroxide oxy (—O—), S(O)₀₋₂, —C(R⁸)—, or amine (—NR⁸—), provided that at least one Z¹ is non-peroxide oxy (—O—), thio (—S—), sulfinyl (—SO—), sulfonyl (—S(O)₂—) or amine (—NR⁸—);

each R⁸ is independently hydrogen, (C₁-C₈)alkyl, (C₁-C₈)alkenyl, (C₃-C₈)cycloalkyl, (C₃-C₈)cycloalkyl(C₁-C₈)alkylene, (C₃-C₈)cycloalkenyl, (C₃-C₈)cycloalkenyl(C₁-C₉)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R^{a})—;

wherein any of the alkyl, cycloalkyl, heterocycle, aryl, or heteroaryl, groups of R¹, R², R³, R^{3a}, R⁶, R⁷ and R⁸ is optionally substituted on carbon with one or more (e.g. 1, 2, 3, or 4) substituents selected from the group consisting of halo, —OR^{a}, —SR^{a}, (C₁-C₈)alkyl, cyano, nitro, trifluoromethyl, trifluoromethoxy, (C₃-C₈)cycloalkyl, (C₆-C₁₂)bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)ₚ—, R^{b}R^{c}NS(O)ₚ—, and —N═NR^{b};

wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;

each R^{a}, R^{b} and R^{c} is independently hydrogen, (C₁-C₁₂)alkyl, (C₁-C₈)alkoxy, (C₁-C₉)alkoxy-(C₁-C₁₂)alkylene, (C₃-C₈)cycloalkyl, (C₃-C₈)cycloalkyl-(C₁-C₁₂)alkylene, (C₁-C₈)alkylthio, amino acid, aryl, aryl(C₁-C₈)alkylene, heterocycle, heterocycle-(C₁-C₈)alkylene, heteroaryl, or heteroaryl(C₁-C₈)alkylene;

alternatively R^{b} and R^{c}, together with the nitrogen to which they are attached, form a pyrrolidino, piperidino, morpholino, or thiomorpholino ring;

wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, —(CH₂)ₐOR^{e}, —(CH₂)ₐSR^{e}, (C₁-C₈)alkyl, (CH₂)ₐCN, (CH₂)ₐNO₂, trifluoromethyl, trifluoromethoxy, —(CH₂)ₐCO₂R³, (CH₂)ₐNR^{e}R^{e}, and (CH₂)ₐC(O)NR^{e}R^{e};

R^{d} is hydrogen or (C₁-C₆)alkyl;

R^{e} is independently selected from H and (C₁-C₆)alkyl;

a is 0, 1, or 2;

i is 1 or 2

m is 0 to 8; and

p is 0 to 2;

provided that m is at least 1 when Z is NR⁴R⁵; or

a pharmaceutically acceptable salt thereof.

Specific values listed below for radicals, substituents, and ranges, are for illustration only; they do not exclude other defined values or other values within defined ranges for the radicals and substituents.

For example, specific values include compounds having the formula (Ia):

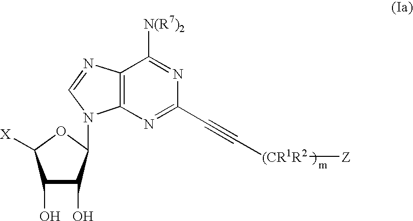

wherein

R¹ is hydrogen, —OH, —CH₂OH, —OMe, —OAc, —NH₂, —NHMe, —NMe₂ or —NHAc;

R² is hydrogen, (C₁-C₈)alkyl, cyclopropyl, cyclohexyl or benzyl;

R³ is hydrogen, OH, OMe, OAc, NH₂, NHMe, NMe₂ or NHAc;

CR⁴R⁵ or NR⁴R⁵ is optionally substituted with 0-2 R⁶ groups and is cyclopentane, cyclohexane, piperidine, dihydro-pyridine, tetrahydro-pyridine, pyridine, piperazine, tetrahydro-pyrazine, dihydro-pyrazine, pyrazine, dihydro-pyrimidine, tetrahydro-pyrimidine, hexahydro-pyrimidine, pyrazine, imidazole, dihydro-imidazole, imidazolidine, pyrazole, dihydro-pyrazole, and pyrazolidine;

alternatively, the ring CR⁴R⁵ or NR⁴R⁵ is optionally substituted with 0-4 (e.g., 0 to 2) R⁶ groups and is selected from the group consisting of:

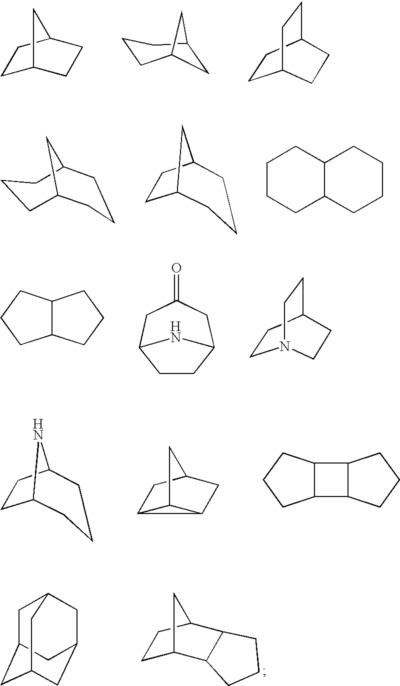

R⁶ is hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;

R^{a}, R^{b} and R^{c} are independently hydrogen, (C₃-C₄)-cycloalkyl, (C₁-C₈)alkyl, aryl or aryl(C₁-C₈)alkylene;

each R⁷ is independently hydrogen, alkyl (e.g., C₁-C₈alkyl), aryl, aryl(C₁-C₈)alkylene or heteroaryl(C₁-C₈)alkylene;

R⁸ is methyl, ethyl, propyl, 2-propenyl, cyclopropyl, cyclobutyl, cyclopropylmethyl, —(CH₂)₂CO₂CH₃, or —(CH₂)₂₋₃OH;

X is —CH₂OR^{a}, —CO₂R^{a}, —CH₂OC(O)R^{a}, or C(O)NR^{b}R^{c};

alternatively X is selected from:

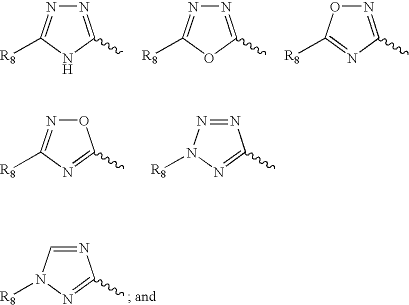

m is 0, 1 or 2;

or a pharmaceutically acceptable salt thereof.

Additional specific values include compounds having the formula (Ia), wherein:

R¹ is hydrogen, OH, OMe, or NH₂;

R² is hydrogen, methyl, ethyl or propyl;

R³ is hydrogen, OH, OMe, or NH₂;

the ring CR⁴R⁵ or NR⁴R⁵ is selected from the group consisting of:

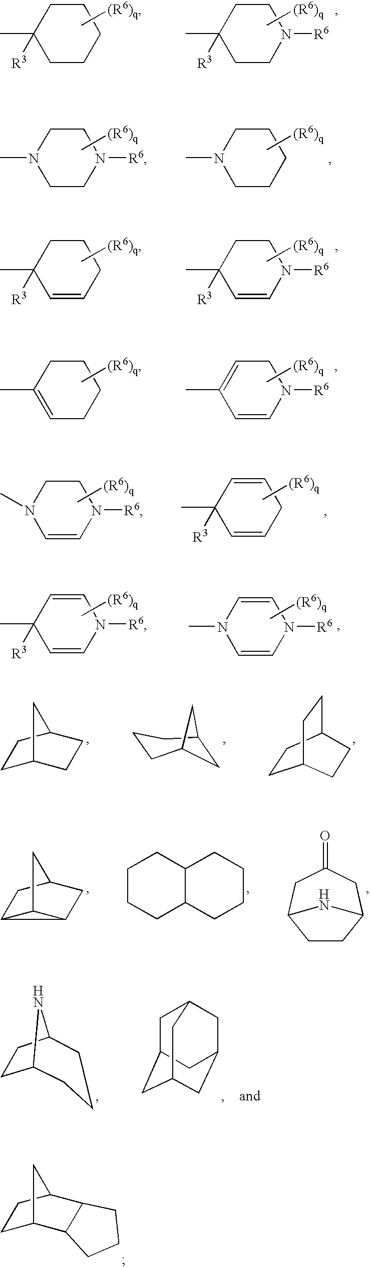

where q is from 0 to 4 (e.g., 0-2);

R⁶ is hydrogen, (C₁-C₈)alkyl, —OR^{a}, —CO₂R^{a}, R^{a}C(═O)—, R^{a}C(═O)O—, R^{b}R^{c}N—, R^{b}R^{c}NC(═O)—, or aryl;

R^{a} and R^{b} are independently hydrogen, methyl, ethyl, propyl, butyl, ethylhexyl, cyclopropyl, cyclobutyl, phenyl or benzyl;

N(R⁷)₂ is amino, methylamino, dimethylamino; ethylamino; pentylamino, diphenylethylamino, (pyridinylmethyl)amino, (pyridinyl)(methyl)amino, diethylamino or benzylamino;

R⁸ is methyl, ethyl, propyl, or cyclopropyl;

X is —CH₂OR^{a} or —C(O)NR^{b}R^{c};

alternatively, X is selected from:

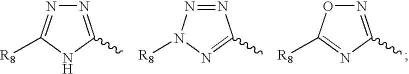

or a pharmaceutically acceptable salt thereof.

Additional specific values include compounds having the formula (Ia), wherein:

R¹ is hydrogen, OH, or NH₂;

R² is hydrogen or methyl;

R³ is hydrogen, OH, or NH₂;

the ring CR⁴R⁵ or NR⁴R⁵ is selected from the group consisting of:

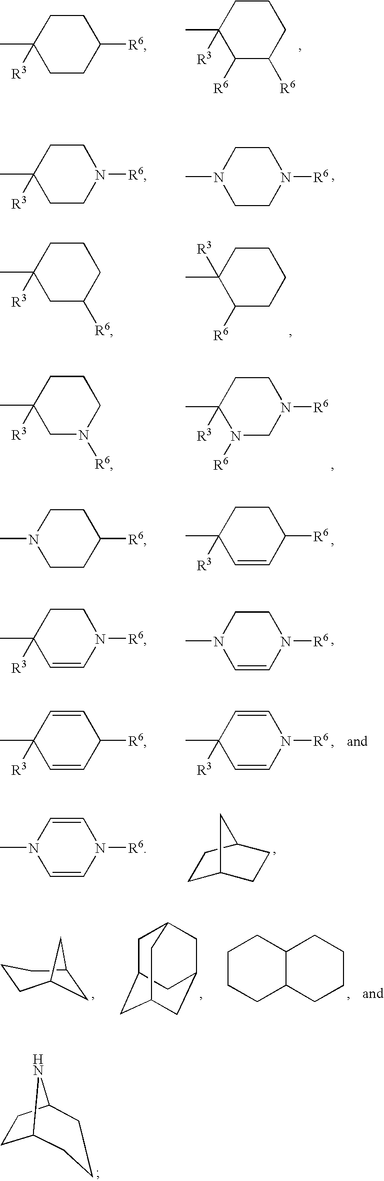

where q is from 0 to 2;

R⁶ is hydrogen, methyl, ethyl, t-butyl, phenyl, —CO₂R^{a}—CONR^{b}R^{c}, or R^{a}C(═O)—;

R^{b} is H;

R^{a} is methyl, ethyl, propyl, butyl, pentyl, ethylhexyl cyclopropyl, and cyclobutyl;

—N(R⁷)₂ is amino, methylamino, dimethylamino; ethylamino; diethylamino or benzylamino;

or a pharmaceutically acceptable salt thereof.

Additional specific values include compounds having the formula (Ia), wherein:

R¹ is hydrogen or OH;

R² is hydrogen;

R³ is hydrogen or OH;

the ring CR⁴R⁵ or NR⁴R⁵ is selected from the group consisting of:

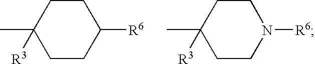

R⁶ is hydrogen, methyl, ethyl, —CO₂R^{a}, and —CONR^{b}R^{c};

R^{b} is H;

R^{a} is methyl, ethyl, i-propyl, i-butyl, tert-butyl, and cyclopropyl;

N(R⁷)₂ is amino, or methylamino;

X is —CH₂OH,

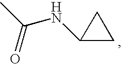

### (O)NHCH₃, or —C(O)NHCH₂CH₃;

or a pharmaceutically acceptable salt thereof.

Additional specific values include compounds wherein the ring comprising R⁴, R⁵ and the atom to which they are connected is 2-methyl cyclohexane, 2,2-dimethylcyclohexane, 2-phenylcyclohexane, 2-ethylcyclohexane, 2,2-diethylcyclohexane, 2-tert-butyl cyclohexane, 3-methyl cyclohexane, 3,3-dimethylcyclohexane, 4-methyl cyclohexane, 4-ethylcyclohexane, 4-phenyl cyclohexane, 4-tert-butyl cyclohexane, 4-carboxymethyl cyclohexane, 4-carboxyethyl cyclohexane, 3,3,5,5-tetramethyl cyclohexane, 2,4-dimethyl cyclopentane, 4-cyclohexanecarboxylic acid, 4-cyclohexanecarboxylic acid esters, 4-methyloxyalkanoyl-cyclohexane, 4-piperidine-1-carboxylic acid methyl ester, 4-piperidine-1-carboxylic acid tert-butyl ester 4-piperidine, 4-piperazine-1-carboxylic acid methyl ester, 4-piperidine-1-carboxylic acid tert-butylester, 1-piperidine-4-carboxylic acid methyl ester, 1-piperidine-4-carboxylic acid tert-butyl ester, tert-butylester, 1-piperidine-4-carboxylic acid methyl ester, or 1-piperidine-4-carboxylic acid tert-butyl ester, 3-piperidine-1-carboxylic acid methyl ester, 3-piperidine-1-carboxylic acid tert-butyl ester, 3-piperidine, 3-piperazine-1-carboxylic acid methyl ester, 3-piperidine-1-carboxylic acid tert-butylester, 1-piperidine-3-carboxylic acid methyl ester, or 1-piperidine-3-carboxylic acid tert-butyl ester; or a pharmaceutically acceptable salt thereof.

Additional specific values include compounds having the formula (Ia), wherein:

R¹ is hydrogen or OH;

R² is hydrogen;

R³ is hydrogen or OH;

the ring CR⁴R⁵ or NR⁴R⁵ is selected from the group consisting of:

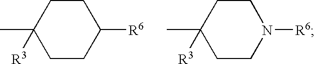

R⁶ is —CO₂R^{a};

R^{a} is (C₁-C₈)alkoxy, (C₃-C₆)cycloalkyl, (C₃-C₆)cycloalkyl-(C₁-C₃)alkylene, heterocycle, or heterocycle-(C₁-C₃)alkylene;

wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, OR^{e}, (C₁-C₄)alkyl, —CN, NO₂, trifluoromethyl, trifluoromethoxy, CO₂R³, NR^{e}R^{e}, and C(O)NR^{e}R^{e}; and,

R^{e} is independently selected from H and (C₁-C₄)alkyl.

Exemplary compounds that are useful in the present invention are shown in Table A below.

TABLE A

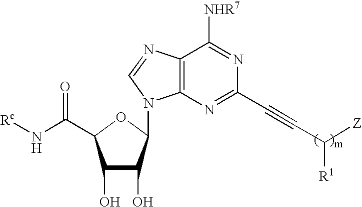

| Ex. | | | |
|---|---|---|---|
| # | R^{c} | R⁷ | -(R¹)ₘ-Z |
| 1. | Et | H | 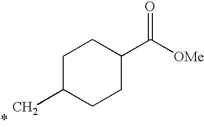 |
| 2. | Et | H | 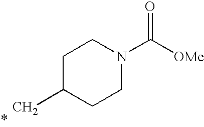 |
| 3. | cPr | H | 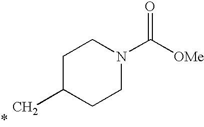 |
| 4. | Et | H | 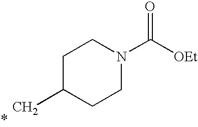 |
| 5. | cPr | H | 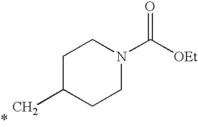 |
| 6. | Et | H | 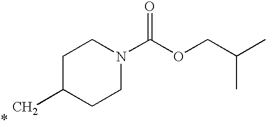 |
| 7. | cPr | H | 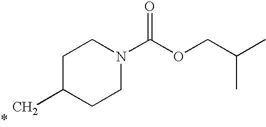 |
| 8. | Et | H | 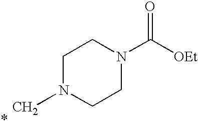 |
| 9. | Et | H | 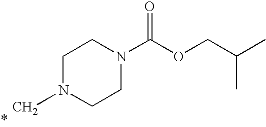 |
| 10. | Et | H | 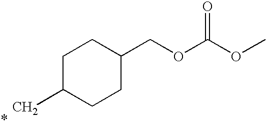 |
| 11. | Et | H | 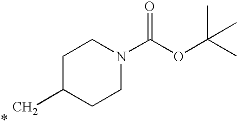 |
| 12. | cPr | H | 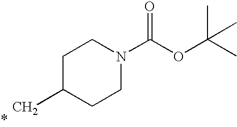 |
| 13. | Et | H | 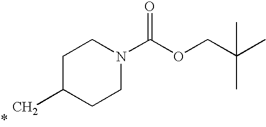 |
| 14. | cPr | H | 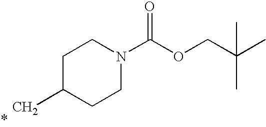 |
| 15. | Et | H | 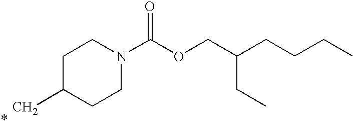 |
| 16. | cPr | H | 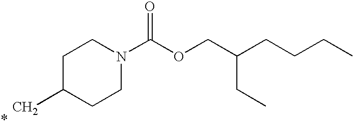 |
| 17 | cPr | H | 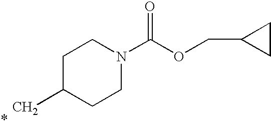 |
| 18. | Et | H | 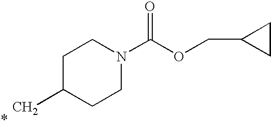 |
| 19. | cPr | H | 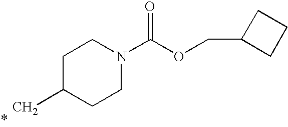 |
| 20. | Et | H | 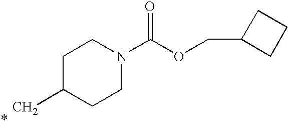 |
| 21. | cPr | H | 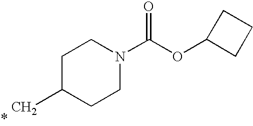 |
| 22. | Et | H | 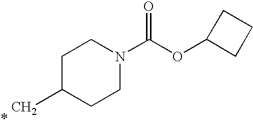 |
| 23 | Et | H | 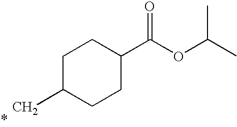 |
| 24. | cPr | H | 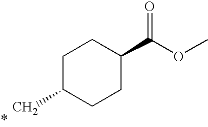 |
| 25. | Et | H | 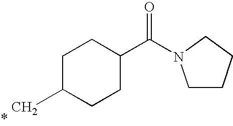 |
| 26. | Et | H | 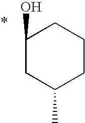 |
| 27. | Et | H | 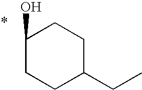 |
| 28. | Et | H | 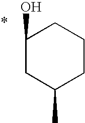 |
| 29. | Et | H | 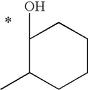 |
| 30. | Et | H | 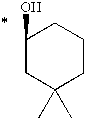 |
| 31. | cPr | H | 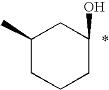 |
| 32. | Et | H | 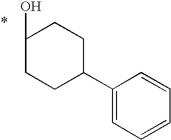 |
| 33. | Et | H | 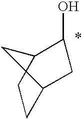 |
| 34. | cPr | H | 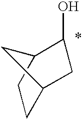 |
| 35. | cPr | H | 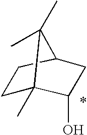 |
| 36. | Et | H | 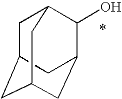 |
| 37. | cPr | H | 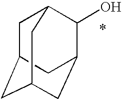 |
| 38. | Et | H | 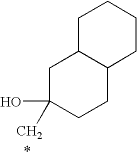 |
| 39. | cPr | H | 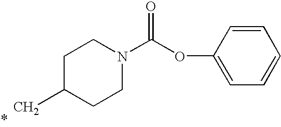 |
| 40. | Et | H | 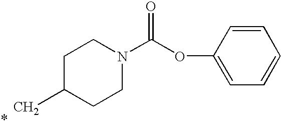 |
| 41. | cPr | H | 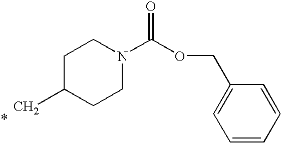 |
| 42. | Et | H | 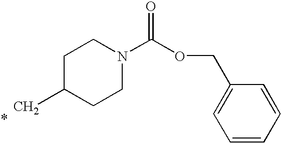 |

wherein * signifies the point of attachment.

Further examples of agonists of A_{2A} adenosine receptors that are useful in the practice of the present invention include compounds having the formula II or a stereoisomer or pharmaceutically acceptable salt thereof:

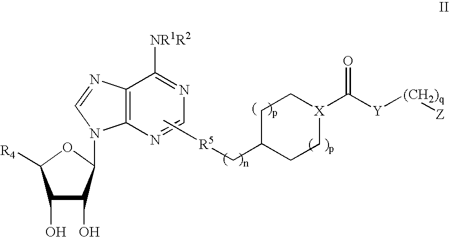

wherein

R¹ and R² independently are selected from the group consisting of H, (C₁-C₈)alkyl, (C₃-C₈)cycloalkyl, (C₃-C₈)cycloalkyl(C₁-C₈)alkylene, aryl, aryl(C₁-C₈)alkylene, heteroaryl, heteroaryl(C₁-C₈)alkylene-, diaryl(C₁-C₈)alkylene, and diheteroaryl(C₁-C₈)alkylene, wherein the aryl and heteroaryl rings are optionally substituted with 1-4 groups independently selected from fluoro, chloro, iodo, bromo, methyl, trifluoromethyl, and methoxy;

each R independently is selected from the group consisting of H, C₁-C₄ alkyl, cyclopropyl, cyclobutyl, and (CH₂)ₐcyclopropyl;

X is CH or N, provided that when X is CH then Z cannot be substituted with halogen, C₁-C₆ alkyl, hydroxyl, amino, or mono- or di-(C₁-C₆-alkyl)amino;

Y is selected from the group consisting of O, NR¹, —(OCH₂CH₂O)ₘCH₂—, and —(NR¹CH₂CH₂O)ₘCH₂—, provided that when Y is O or NR¹, then at least one substituent is present on Z;

Z is selected from the group consisting of 5-membered heteroaryl, 6-membered aryl, 6-membered heteroaryl, carbocyclic biaryl, and heterocyclic biaryl, wherein the point of attachment of Y to Z is a carbon atom on Z, wherein Z is substituted with 0-4 groups independently selected from the group consisting of F, Cl, Br, I, (C₁-C₄)alkyl, —(CH₂)ₐOR³, —(CH₂)ₐNR³R³, —NHOH, —NR³NR³R³, nitro, —(CH₂)ₐCN, —(CH₂)ₐCO₂R³, —(CH₂)ₐCONR³R³, trifluoromethyl, and trifluoromethoxy;

alternatively, Y and Z together form an indolyl, indolinyl, isoindolinyl, tetrahydroisoquinolinyl, or tetrahydroquinolinyl moiety wherein the point of attachment is via the ring nitrogen and wherein said indolyl, indolinyl, isoindolinyl, tetrahydroisoquinolinyl, or tetrahydroquinolinyl moiety, which is substituted with 0-4 groups independently selected from the group consisting of F, Cl, Br, I, C₁-C₄ alkyl, —(CH₂)ₐOR³, —(CH₂)ₐNR³R³, —NHOH, —NR³NR³R³, NO₂, —(CH₂)ₐCN, —(CH₂)ₐCO₂R³, —(CH₂)ₐCONR³R³, CF₃, and OCF₃;

R³ is independently selected from the group consisting of H, (C₁-C₆)alkyl, cycloalkyl, aryl, and heteroaryl;

R⁴ is selected from the group consisting of CH₂OR, C(O)NRR, and CO₂R;

R⁵ is selected from the group consisting of CH₂CH₂, CH═CH, and C≡C;

a is selected from 0, 1, and 2;

m is selected from 1, 2, and 3;

n is selected from 0, 1, and 2;

each p independently is selected from 0, 1, and 2; and,

q is selected from 0, 1, and 2.

Additional specific values include compounds having the formula IIa or a pharmaceutically acceptable salt thereof:

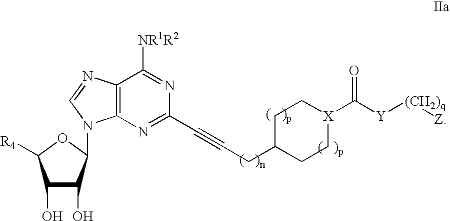

Additional specific values include compounds having the formula IIb or a pharmaceutically acceptable salt thereof:

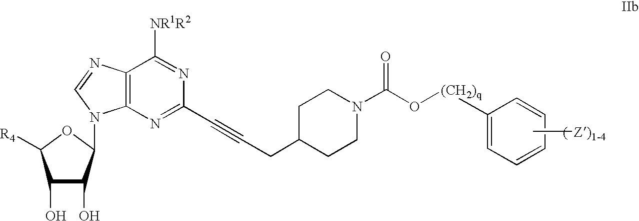

wherein:

each Z′ is independently selected from the group consisting F, Cl, Br, I, C₁-C₄ alkyl, (CH₂)ₐOR³, —(CH₂)ₐNR³R³, —NHOH, —NR³NR³R³, NO₂, —(CH₂)ₐCN, —(CH₂)ₐCO₂R³, —(CH₂)ₐCONR³R³, CF₃, and OCF₃.

Additional specific values include compounds wherein R is selected from H, methyl, ethyl or cyclopropyl.

Additional specific values include compounds having the formula IIc or a pharmaceutically acceptable salt thereof:

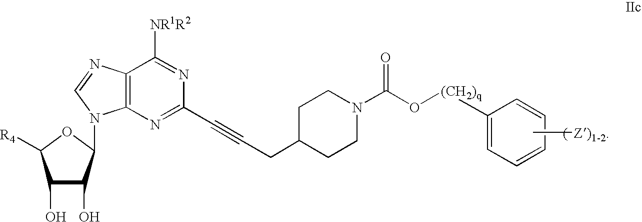

Additional specific values include compounds wherein Z¹ is selected from the group consisting of F, Cl, methyl, OR³, NO₂, CN, NR³R³ and CO₂R³.

Additional specific values include compounds wherein R³ is methyl or hydrogen.

Additional exemplary compounds that are useful in the present invention are shown in Table B below.

TABLE B

| | |
|---|---|
| | i |
| 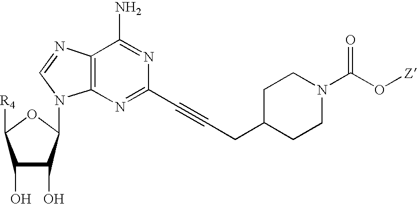 | |
| | ii |
| 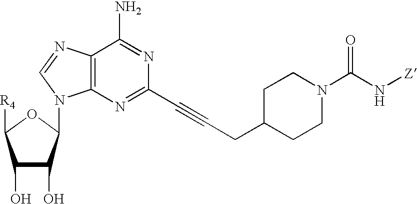 | |
| | iii |
| 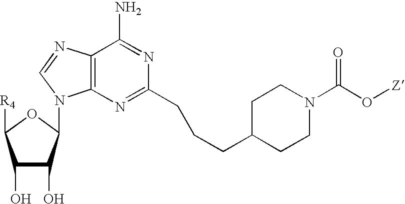 | |

| Ex. # | R⁴ | Z′ |
|---|---|---|
| 1 | C | 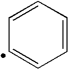 |
| 2 | C | 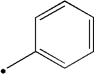 |
| 3 | C | 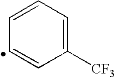 |
| 4 | A | 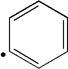 |
| 5 | C | 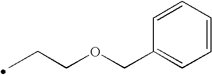 |
| 6 | A | 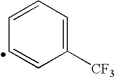 |
| 7 | A | 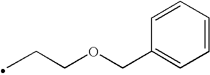 |
| 8 | C | 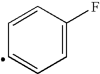 |
| 9 | C | 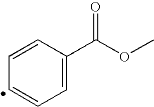 |
| 10 | C | 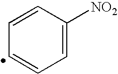 |
| 11 | A | 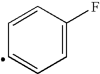 |
| 12 | A | 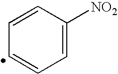 |
| 13 | A | 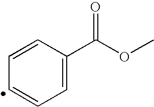 |
| 14 | C | 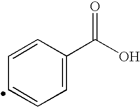 |
| 15 | B | 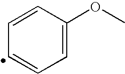 |
| 16 | B | 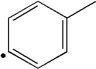 |
| 17 | C | 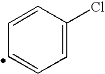 |
| 18 | C | 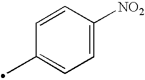 |
| 19 | B | 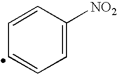 |
| 20 | C | 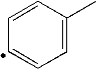 |
| 21 | C | 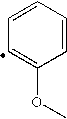 |
| 22 | C | 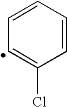 |
| 23 | C | 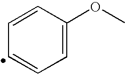 |
| 24 | B | 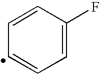 |
| 25 | B | 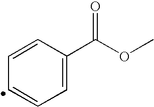 |
| 26 | B | 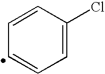 |
| 27 | A | 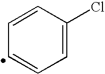 |
| 28 | A | 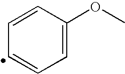 |
| 29 | A | 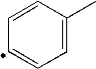 |
| 30 | A | 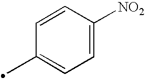 |
| 31 | B | 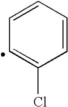 |
| 32 | B | 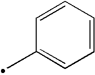 |
| 33 | B | 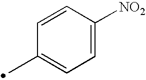 |
| 34 | B | 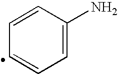 |
| 35 | A | 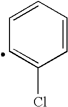 |
| 36 | A | 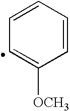 |
| 37(iii) | B | 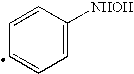 |
| 38(iii) | C | 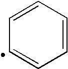 |
| 39(iii) | C | 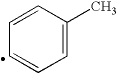 |
| 40(iii) | C | 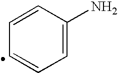 |
| 41(iii) | C | 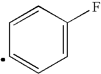 |
| 42 | C | 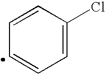 |
| 43(ii) | C | 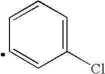 |
| 44(ii) | A | 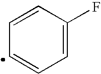 |
| 45(ii) | A | 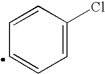 |
| 46(ii) | A | 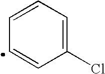 |
| 47(ii) | C | 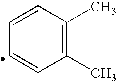 |
| 48(ii) | C | 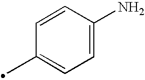 |
| 49 | B | 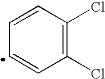 |
| 50 | B | 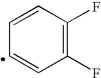 |
| 51 | C | 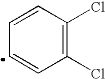 |
| 52 | C | 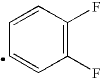 |
| 53 | A | 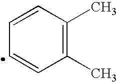 |
| 54 | A | 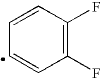 |
| 55 | A | 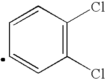 |
| 56 | C | 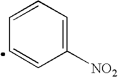 |
| 57 | C | 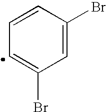 |

wherein • signifies the point of attachment.
R⁴ = A: CH₂OH; B: C(O)NEthyl; C: C(O)C(O)NCyclopropyl.
Compounds are formula (i), unless indicated.

Additional specific values include compounds having the formula (Ib)-(Id) or a pharmaceutically acceptable salt thereof:

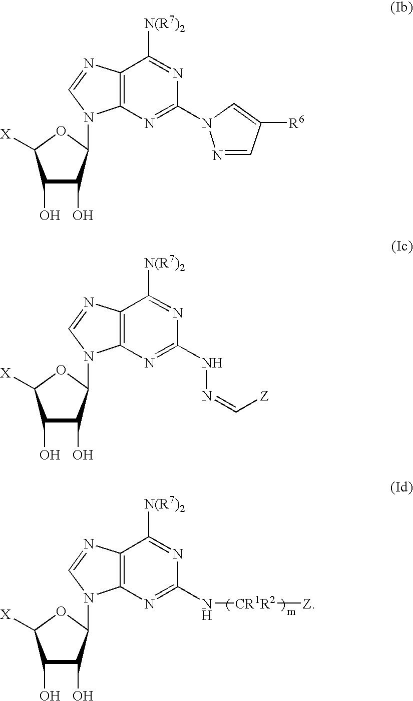

Additional examples of A_{2A} adenosine receptor agonists that can be useful in the present invention include compounds of formula 4:

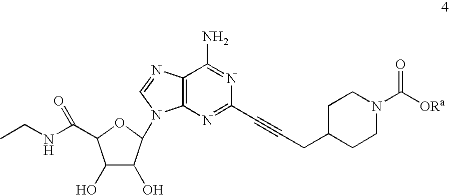

wherein R^{a} is methyl, ethyl, propyl, isopropyl, isobutyl, or t-butyl.

Additional examples of A_{2A} adenosine receptor agonists that can be useful in the present invention include those described in U.S. Pat. No. 6,232,297 and in U.S. Patent Application No. 2003/0186926 A1, which are incorporated herein by reference.

Further examples of compounds expected that can be useful in the present invention include compounds of formula (IA)

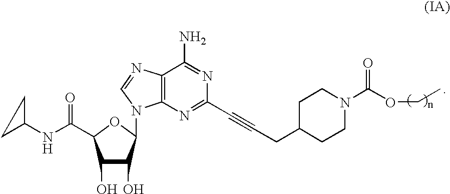

In formula (IA) n is 0, 1, 2, 3, 4, 5, 6, 7, 8, 9, 10, 11, 12, 13, 14, 15, 16, 17, or 18. In another group of specific compounds n is, 5, 6, 7, 8, 9, 10, 11, 12, 13, 14, 15, 16, 17, or 18.

Additional examples of A_{2A} adenosine receptor agonists that can be useful in the present invention include compounds of formula (IB)

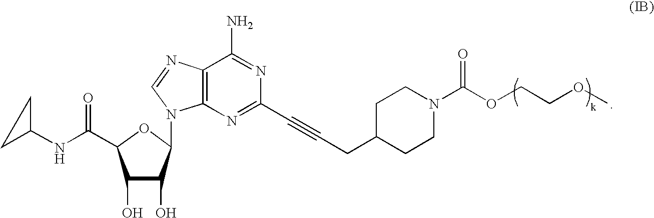

In formula (IB) k is 1, 2, 3, 4, 5, 6, 7, 8, 9, 10, 11, 12, 13, 14, 15, 16, 17, or 18.

Additional examples of A_{2A} adenosine receptor agonists that can be useful in the present invention include compounds of formula (IC)

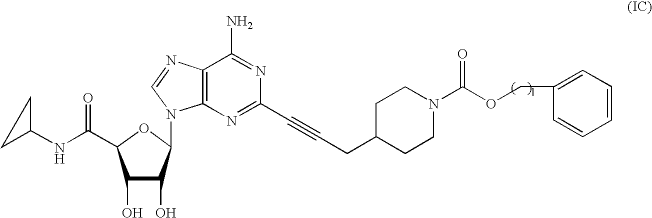

wherein 1 is 0, 1, 2, 3, or 4.

Other specific compounds of the invention include

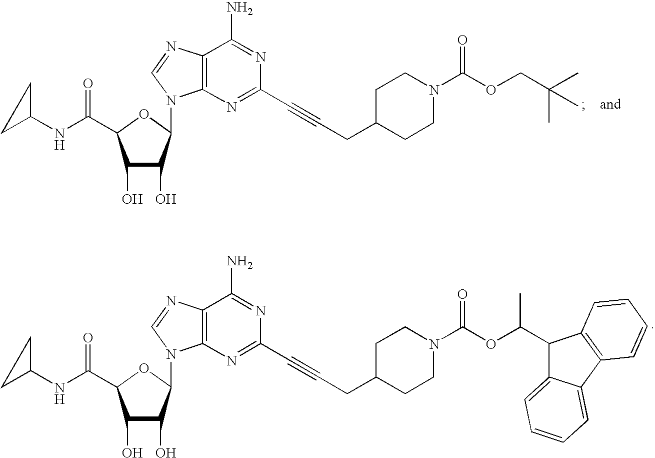

Additional examples of compounds that can be useful in the present invention are illustrated in Tables 1, 2, and 3 below:

TABLE 1

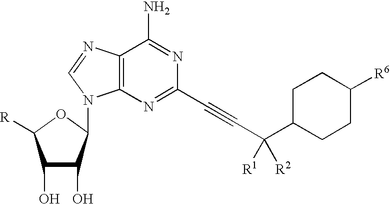

| Compound | R | R¹ | R² | R⁶ |
|---|---|---|---|---|
| ATL2037 | NECA | H | H | CH₂OH |
| MP9056 | NECA | OH | H | CH₂OH |
| ATL146a | NECA | H | H | CO₂H |
| MP9057 | NECA | OH | H | CO₂H |
| ATL146e | NECA | H | H | CO₂Me |
| MP9058 | NECA | OH | H | CO₂Me |
| JR2145 | CH₂OH | H | H | CO₂Me |
| MP9059 | CH₂OH | OH | H | CO₂Me |
| ATL193 | NECA | H | H | CH₂OAc |
| MP9060 | NECA | OH | H | CH₂OAc |
| JR2147 | CH₂OH | H | H | CH₂OAc |
| MP9061 | CH₂OH | OH | H | CH₂OAc |
| JR3023 | NECA | H | H | CH₂N(CH₃)₂ |
| MP9062 | NECA | OH | H | CH₂N(CH₃)₂ |
| JR3021 | NECA | H | H | COOCH₂CH₂NHBoc |
| MP9063 | NECA | OH | H | COOCH₂CH₂NHBoc |
| JR3033 | NECA | H | H | COOCH₂CH₂NH₂ |
| MP9064 | NECA | OH | H | COOCH₂CH₂NH₂ |
| JR3037 | NECA | H | H | CONHCH₂CH₃ |
| MP9065 | NECA | OH | H | CONHCH₂CH₃ |
| JR3055 | NECA | H | H | CONH₂ |
| MP9072 | NECA | OH | H | CONH₂ |
| JR3065 | NECA | H | H | CONHMe |
| MP9066 | NECA | OH | H | CONHMe |
| JR3067B | NECA | H | H | Me, cis CO₂Me |
| MP9067 | NECA | OH | H | Me, cis CO₂Me |
| JR3067A | NECA | H | H | Me, trans CO₂Me |
| MP9068 | NECA | OH | H | Me, trans CO₂Me |
| JR3087 | NECA | H | H | CH₂CH₃ |
| MP9069 | NECA | OH | H | CH₂CH₃ |
| JR3159A | NECA | OH | H | H |
| JR3159B | NECA | OH | H | H |
| JR3119 | NECA | H | H | COCH₃ |
| MP9070 | NECA | OH | H | COCH₃ |
| JR3121 | NECA | H | H | CHCH₃(OH) |
| MP9071 | NECA | OH | H | CHCH₃(OH) |
| JR3139 | NECA | OH | C₆H₁₁ | H |

NECA = CH₃CH₂N(H)C(O)—

TABLE 2

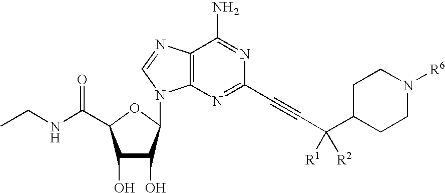

| Compound | R¹ | R² | R⁶ |
|---|---|---|---|
| JR3261 | H | H | H |
| JR3259 | H | H | CO₂tBu |
| JR3269 | H | H | CO₂Et |
| JR4011 | H | H | CO₂iBu |
| JR4009 | H | H | CO₂iPr |
| JR4007 | H | H | COMe |
| JR4051 | H | H | COC(CH₃)₃ |
| JR4047 | H | H | COCH₂(CH₃)₃ |
| MP9047 | H | H | COCH₃ |
| MP9048 | H | H | C(O)N(CH₃)₂ |
| MP9049 | H | H | C(O)N(CH₃)Et |
| MP9050 | H | H | C(O)N(CH₃)iPr |
| MP9051 | H | H | C(O)N(CH₃)iBu |
| MP9052 | H | H | C(O)NH(CH₃) |
| MP9053 | H | H | C(O)NH(Et) |
| MP9054 | H | H | C(O)NH(iPr) |
| MP9055 | H | H | C(O)NH(iBu) |
| TX3261 | OH | H | H |
| TX3259 | OH | H | CO₂tBu |
| TX3269 | OH | H | CO₂Et |
| TX4011 | OH | H | CO₂iBu |
| TX4009 | OH | H | CO₂iPr |
| TX4007 | OH | H | COMe |
| TX4051 | OH | H | COC(CH₃)₃ |
| TX4047 | OH | H | COCH₂(CH₃)₃ |
| TX9047 | OH | H | COCH₃ |
| TX9048 | OH | H | C(O)N(CH₃)₂ |
| TX9049 | OH | H | C(O)N(CH₃)Et |
| TX9050 | OH | H | C(O)N(CH₃)iPr |
| TX9051 | OH | H | C(O)N(CH₃)iBu |
| TX9052 | OH | H | C(O)NH(CH₃) |
| TX9053 | OH | H | C(O)NH(Et) |
| TX9054 | OH | H | C(O)NH(iPr) |
| TX9055 | OH | H | C(O)NH(iBu) |

TABLE 3

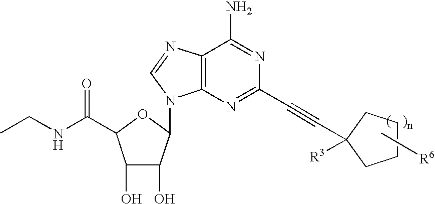

| Compound | n | R³ | R⁶ |
|---|---|---|---|
| JR3135 | 1 | OH | H |
| JR3089 | 2 | OH | H |
| JR3205 | 2 | NH₂ | H |
| JR3177A | 2 | OH | 2-CH₃ |
| JR3177B | 2 | OH | 2-CH₃ |
| JR3181A | 2 | OH | 2-CH₃ |
| JR3181B | 2 | OH | 2-CH₃ |
| JR3227 | 2 | OH | 2-C(CH₃)₃ |
| JR9876 | 2 | OH | 2-C₆H₅ |
| JR3179 | 2 | OH | 3-CH₃ |
| JR3221 | 2 | OH (R) | 3-CH₃ (R) |
| ATL203 | 2 | OH (S) | 3-CH₃ (R) |
| MP9041 | 2 | OH (R) | 3-CH₃ (S) |
| MP9042 | 2 | OH (S) | 3-CH₃ (S) |
| JR3201B | 2 | OH | 3-(CH₃)₂ |
| MP9043 | 2 | OH (R) | 3-CH₂CH₃ (R) |
| MP9044 | 2 | OH (S) | 3-CH₂CH₃ (R) |
| MP9045 | 2 | OH (R) | 3-CH₂CH₃ (S) |
| MP9046 | 2 | OH (S) | 3-CH₂CH₃ (S) |
| JR3163 | 2 | OH | 3-(CH₃)2, 5-(CH₃)₂ |
| JR9875 | 2 | OH | 4-CH₃ |
| JR3149 | 2 | OH | 4-C₂H₅ |
| JR3203 | 2 | OH | 4-C(CH₃)₃ |
| JR3161 | 2 | OH | 4-C₆H₅ |

Additional examples of A_{2A} adenosine receptor agonists that can be useful in the present invention include compounds of formula (II):

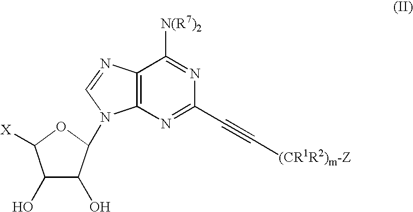

wherein Z is CR³R⁴R⁵; each R¹, R² and R³ is hydrogen; R⁴ and R⁵ together with the carbon atom to which they are attached form a cycloalkyl ring having 3, 4, 5, 6, 7, 8, 9 or 10 ring atoms; and

wherein the ring comprising R⁴ and R⁵ is substituted with —(CH₂)₀₋₆—Y; where Y is —CH₂OR^{a}, —CO₂R^{a}, —OC(O)R^{a}, —CH₂OC(O)R^{a}, —C(O)NR^{b}R^{c}, —CH₂SR^{a}, —C(S)OR^{a}, —OC(S)R^{a}, —CH₂OC(S)R^{a} or C(S)NR^{b}R^{c} or —CH₂N(R^{b})(R^{c});

each R⁷ is independently hydrogen, (C₁-C₈)alkyl, (C₃-C₈)cycloalkyl, aryl or aryl(C₁-C₈)alkylene;

X is —CH₂OR^{a}, —CO₂R^{a}, —CH₂OC(O)R^{a}, —C(O)NR^{b}R^{c}, —CH₂SR^{a}, —C(S)OR^{a}, —CH₂OC(S)R^{a}, C(S)NR^{b}R^{c}C or —CH₂N(R^{b})(R^{c});

each R^{a}, R^{b} and R^{c} is independently hydrogen, (C₁-C₈)alkyl, or (C₁-C₈)alkyl substituted with 1-3 (C₁-C₈)alkoxy, (C₃-C₈)cycloalkyl, (C₁-C₈)alkylthio, amino acid, aryl, aryl(C₁-C₈)alkylene, heteroaryl, or heteroaryl(C₁-C₈)alkylene; or R^{b} and R^{c}, together with the nitrogen to which they are attached, form a pyrrolidino, piperidino, morpholino, or thiomorpholino ring; and m is 0 to about 6; or a pharmaceutically acceptable salt thereof.

A specific value for —N(R⁷)₂ is amino, monomethylamino or cyclopropylamino.

A specific value for Z is carboxy- or —(C₁-C₄)alkoxycarbonyl-cyclohexyl(C₁-C₄)alkyl.

A specific value for R^{a} is H or (C₁-C₄)alkyl, i.e., methyl or ethyl.

A specific value for R^{b} is H, methyl or phenyl.

A specific value for R^{c} is H, methyl or phenyl.

A specific value for —(CR¹R²)ₘ— is —CH₂— or —CH₂—CH₂—.

A specific value for X is CO₂R^{a}, (C₂-C₅)alkanoylmethyl or amido.

A specific value for Y is CO₂R^{a}, (C₂-C₅)alkanoylmethyl or amido.

A specific value for m is 1.

Specific compounds that can be useful for practicing the invention are compounds JR3259, JR3269, JR4011, JR4009, JR-1085 and JR4007.

Specific A_{2A} adenosine receptor agonists that can be useful in the present invention having formula (II) include those described in U.S. Pat. No. 6,232,297, which is incorporated herein by reference.

Specific compounds of formula (II) are those wherein each R⁷ is H, X is ethylaminocarbonyl and Z is 4-carboxycyclohexylmethyl (DWH-146a), Z is 4-methoxycarbonylcyclohexylmethyl (DWH-146e), Z is 4-isopropylcarbonyl-cyclohexylmethyl (AB-1), Z is 4-acetoxymethyl-cyclohexylmethyl (JMR-193) or Z is 4-pyrrolidine-1-carbonylcyclohexylmethyl (AB-3).

Additional examples of A_{2A} adenosine receptor agonists that can be useful in the present invention include those depicted below:

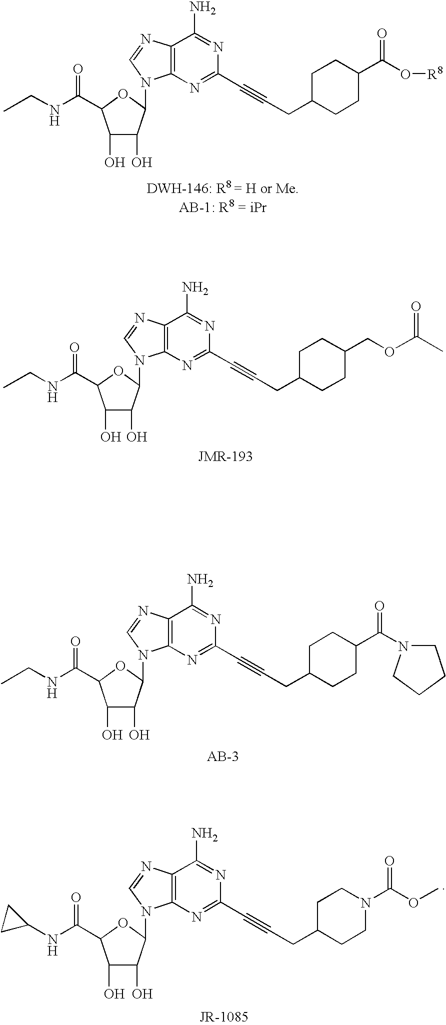

Additional examples of A_{2A} adenosine receptor agonists of formula (II) that can be useful in the present invention include those described in U.S. Pat. No. 6,232,297, which is incorporated herein by reference. These compounds, having formula (II), can be prepared according to the methods described therein.

Another specific group of agonists of A_{2A} adenosine receptors that can be useful in the practice of the present invention include compounds having the general formula (III):

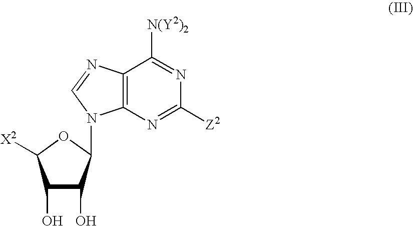

wherein Z² is a group selected from the group consisting of —OR¹², —NR¹³R¹⁴, a —C≡C-Z³, and —NH—N═R¹⁷;

each Y² is individually H, C₁-C₆ alkyl, C₃-C₇ cycloalkyl, phenyl or phenyl C₁-C₃ alkyl;

R¹² is C₁₋₄alkyl; C₁₋₄-alkyl substituted with one or more C₁₋₄-alkoxy groups, halogens (fluorine, chlorine or bromine), hydroxy groups, amino groups, mono(C₁₋₄-alkyl)amino groups, di(C₁₋₄-alkyl)amino groups or C₆₋₁₀-aryl groups wherein the aryl groups may be substituted with one or more halogens (fluorine, chlorine or bromine), C₁₋₄-alkyl groups, hydroxy groups, amino groups, mono(C₁₋₄-alkyl)amino groups or di(C₁₋₄-alkyl)amino groups); or C₆₋₁₀-aryl; or C₆₋₁₀-aryl substituted with one or more halogens (fluorine, chlorine or bromine), hydroxy groups, amino groups, mono(C₁₋₄-alkyl)amino groups, di(C₁₋₄-alkyl)amino groups or C₁₋₄-alkyl groups;

one of R¹³ and R¹⁴ has the same meaning as R¹² and the other is hydrogen; and

R¹⁷ is a group having the formula (i)

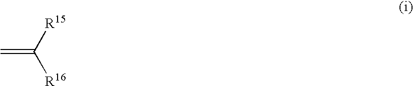

wherein each of R¹⁵ and R¹⁶ independently may be hydrogen, (C₃-C₇)cycloalkyl or any of the meanings of R¹², provided that R¹⁵ and R¹⁶ are not both hydrogen;

X² is CH₂OH, CH₃, CO₂R²⁰ or C(═O)NR²¹R²² wherein R²⁰ has the same meaning as R¹³ and wherein R²¹ and R²² have the same meanings as R¹⁵ and R¹⁶ or R²¹ and R²² are both H;

Z³ has one of the following meanings:

C₆-C₁₀ aryl, optionally substituted with one to three halogen atoms, C₁-C₆ alkyl, C₁-C₆ haloalkyl, C₁-C₆ alkoxy, C₁-C₆ haloalkoxy, C₂-C₆ alkoxycarbonyl, C₂-C₆ alkoxyalkyl, C₁-C₆ alkylthio, thio, CHO, cyanomethyl, nitro, cyano, hydroxy, carboxy, C₂-C₆ acyl, amino C₁-C₃ monoalkylamino, C₂-C₆ dialkylamino, methylenedioxy or aminocarbonyl;

a group of formula —(CH₂)_{q}-Het wherein q is 0 or an integer from 1 to 3 and Het is 5 or 6 membered heterocyclic aromatic or non-aromatic ring, optionally benzocondensed, containing 1 to 3 heteroatoms selected from non-peroxide oxygen, nitrogen or sulphur, linked through a carbon atom or through a nitrogen atom;

C₃-C₇ cycloalkyl optionally containing unsaturation or C₂-C₄ alkenyl;

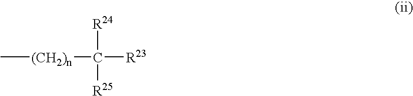

wherein

R²³ is hydrogen, methyl or phenyl;

R²⁴ is hydrogen, C₁-C₆ linear or branched alkyl, C₅-C₆ cycloalkyl or C₃-C₇ cycloalkenyl, phenyl-C₁-C₂-alkyl or R²³ and R²⁴, taken together, form a 5 or 6-membered carbocyclic ring or R²⁵ is hydrogen and R²³ and R²⁴, taken together, form an oxo group or a corresponding acetalic derivative;

R²⁵ is OH, NH₂ dialkylamino, halogen, or cyano; and n is 0 or 1 to 4; or C₁-C₁₆ alkyl, optionally comprising 1-2 double bonds, O, S or NY²;

or a pharmaceutically acceptable salt thereof.

Specific C₆₋₁₀-aryl groups include phenyl and naphthyl.

Additional specific values include compounds wherein in the compound of formula (I), Z² is a group of the formula (iii)

—O—(CH₂)ₙ—Ar (iii)

wherein n is an integer from 1-4, e.g., 2, and Ar is a phenyl group, tolyl group, naphthyl group, xylyl group or mesityl group. In one embodiment, Ar is a para-tolyl group and n=2.

Additional specific values include compounds wherein in the compound of formula (III), Z² is a group of the formula (Iv)

NHN═CHCy (iv)

wherein Cy is a C₃₋₇-cycloalkyl group, such as cyclohexyl or a C₁₋₄ alkyl group, such as isopropyl.

Additional specific values include compounds wherein in the compound of formula (III), Z² is a group of the formula (v)

C≡CZ³ (v)

wherein Z³ is C₃-C₁₆ alkyl, hydroxy C₂-C₆ alkyl or (phenyl) (hydroxymethyl).

Additional examples of compounds of formula (III) include those shown below:

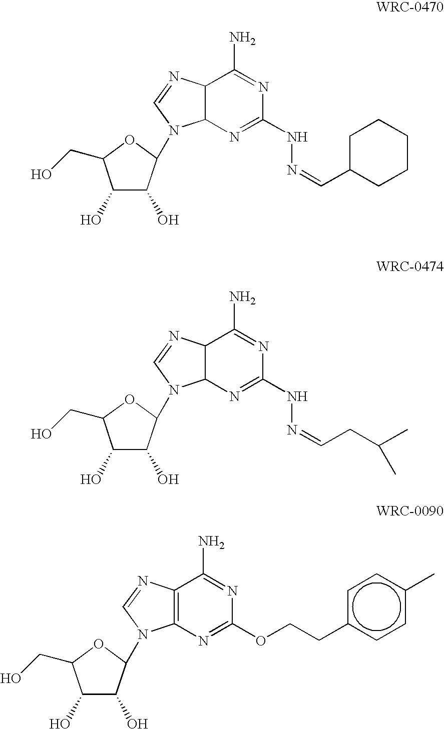

wherein the H on CH₂OH can optionally be replaced by ethylaminocarbonyl. Of these specific examples, WRC-0474-[SHA 211] and WRC-0470 are particularly preferred.

Such compounds may be synthesized as described in: Olsson et al. (U.S. Pat. Nos. 5,140,015 and 5,278,150); Cristalli (U.S. Pat. No. 5,593,975); Miyasaka et al. (U.S. Pat. No. 4,956,345); Hutchinson, A. J. et al., J. Pharmacol. Exp. Ther., 251, 47 (1989); Olsson, R. A. et al., J. Med. Chem., 29, 1683 (1986); Bridges, A. J. et al., J. Med. Chem., 31, 1282 (1988); Hutchinson, A. J. et al., J. Med. Chem., 33, 1919 (1990); Ukeeda, M. et al., J. Med. Chem., 34, 1334 (1991); Francis, J. E. et al., J. Med. Chem., 34, 2570 (1991); Yoneyama, F. et al., Eur. J. Pharmacol., 213, 199-204 (1992); Peet, N. P. et al., J. Med. Chem., 35, 3263 (1992); and Cristalli, G. et al., J. Med. Chem., 35, 2363 (1992); all of which are incorporated herein by reference.

Additional specific values include compounds having formula (III) where Z² is a group having formula (vi):

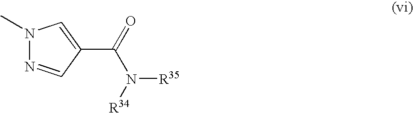

wherein R³⁴ and R³⁵ are independently H, C₁-C₆ alkyl, C₃-C₇ cycloalkyl, phenyl, phenyl C₁-C₃ alkyl or R³⁴ and R³⁵ taken together with the nitrogen atom are a 5- or 6-membered heterocyclic ring containing 1-2 heteroatoms selected from non-peroxide oxygen, nitrogen (N(R¹³)) or sulphur atoms. In one embodiment, one of R³⁴ and R³⁵ is hydrogen and the other is ethyl, methyl or propyl. In another embodiment, one of R³⁴ and R³⁵ is hydrogen and the other is ethyl or methyl.

A specific pyrazole derivative that is expected to be useful in practicing the present invention is a compound having the formula:

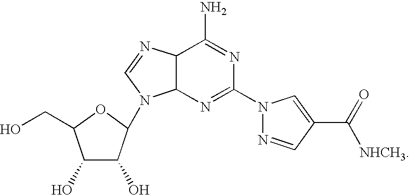

Another specific group of agonists of A_{2A} adenosine receptors that are expected to be useful in the present invention include compounds having the general formula (IV):

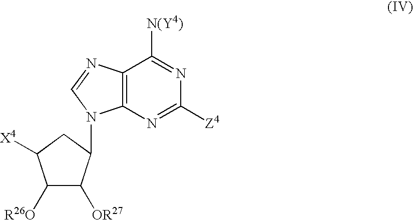

wherein Z⁴ is —N_R²⁹;

R²⁸ is hydrogen or (C₁-C₄) alkyl; and R²⁹ is
a) (C₁-C₄) alkyl;
b) (C₁-C₄) alkyl substituted with one or more (C₁-C₄) alkoxy, halogen, hydroxy, amino, mono((C₁-C₄) alkyl)amino, di((C₁-C₄) alkyl)amino or (C₆-C₁₀) aryl wherein aryl is optionally substituted with one or more halogen, hydroxy, amino, (C₁-C₄)alkyl, R³⁰OOC—((C₁-C₄)alkyl)-, R³¹R³²NC(═O)—((C₁-C₄)alkyl)-, mono((C₁-C₄)alkyl)amino or di((C₁-C₄)alkyl)amino;
c) (C₆-C₁₀)aryl; or
d) (C₆-C₁₀)aryl substituted with one or more halogen, hydroxy, amino, mono((C₁-C₄)alkyl)amino, di((C₁-C₄)alkyl)amino or (C₁-C₄)alkyl;

wherein each Y⁴ is individually H, (C₁-C₆)alkyl, (C₃-C₇)cycloalkyl, phenyl or phenyl(C₁-C₃)alkyl; and X⁴ is —C(═O)NR³¹R³², —COOR³⁰, or —CH₂OR³⁰;

wherein each of R³¹ and R³² are independently hydrogen; C₃₋₇-cycloalkyl; (C₁-C₄)alkyl; (C₁-C₄)alkyl substituted with one or more (C₁-C₄)alkoxy, halogen, hydroxy, —COOR³³, amino, mono((C₁-C₄)alkyl)amino, di((C₁-C₄)alkyl)amino or (C₆-C₁₀)aryl wherein aryl is optionally substituted with one or more halogen, (C₁-C₄)alkyl, hydroxy, amino, mono((C₁-C₄) alkyl)amino or di((C₁-C₄) alkyl)amino; (C₆-C₁₀)aryl; or (C₆-C₁₀)aryl substituted with one or more halogen, hydroxy, amino, mono((C₁-C₄)alkyl)amino, di((C₁-C₄)alkyl)amino or (C₁-C₄)alkyl;

R²⁶ and R²⁷ independently represent hydrogen, lower alkanoyl, lower alkoxy-lower alkanoyl, aroyl, carbamoyl or mono- or di-lower alkylcarbamoyl; and R³⁰ and R³³ are independently hydrogen, (C₁-C₄)alkyl, (C₆-C₁₀)aryl or (C₆-C₁₀)aryl((C₁-C₄)alkyl); or a pharmaceutically acceptable salt thereof.

Additional specific values include compounds wherein at least one of R²³ and R²⁹ is (C₁-C₄)alkyl substituted with one or more (C₁-C₄)alkoxy, halogen, hydroxy, amino, mono((C₁-C₄)alkyl)amino, di((C₁-C₄)alkyl)amino or (C₆-C₁₀)aryl wherein aryl is optionally substituted with one or more halogen, hydroxy, amino, (C₁-C₄)alkyl, R³⁰OOC(C₁-C₄)alkyl, mono((C₁-C₄)alkyl)amino or di((C₁-C₄)alkyl)amino. Additional specific values include compounds wherein at least one of R³¹ and R³² is C₁₋₄-alkyl substituted with one or more (C₁-C₄)alkoxy, halogen, hydroxy, amino, mono((C₁-C₄)alkyl)amino, di((C₁-C₄)alkyl)amino or C₆₋₁₀-aryl wherein aryl is optionally substituted with one or more halogen, hydroxy, amino, (C₁-C₄)alkyl, R³⁰OOC—(C₁-C₄)alkylene-, mono((C₁-C₄)alkyl)amino or di((C₁-C₄)alkyl)amino. Additional specific values include compounds wherein at least one of R²⁸ and R²⁹ is C₆₋₁₀-aryl substituted with one or more halogen, hydroxy, amino, mono((C₁-C₄)alkyl)amino, di((C₁-C₄)alkyl)amino or (C₁-C₄)alkyl.

Additional specific values include compounds wherein at least one of R³¹ and R³² is C₆₋₁₀-aryl substituted with one or more halogen, hydroxy, amino, mono((C₁-C₄)alkyl)-amino, di((C₁-C₄)alkyl)amino or (C₁-C₄)alkyl.

Additional specific values include compounds wherein R³¹ is hydrogen and R³² is (C₁-C₄)alkyl, cyclopropyl or hydroxy-(C₂-C₄)alkyl. A specific R²⁸ group is (C₁-C₄)alkyl substituted with (C₆-C₁₀)aryl, that is in turn substituted with R³⁰O(O)C—(C₁-C₄)alkylene-.

A specific compound having formula (IV) is:

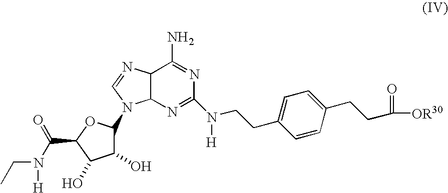

wherein R³⁰ is hydrogen, methyl, ethyl, n-propyl or isopropyl. One embodiment provides a compound wherein the R³⁰ group is methyl or ethyl. In one embodiment, the R³⁰ group is methyl.

Two compounds that can be useful in practicing the present invention have the formula:

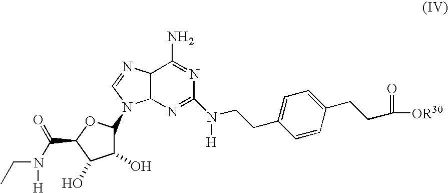

wherein R³⁰ is hydrogen (acid, CGS21680) or wherein R³⁰ is methyl (ester, JR2171).

The compounds of the invention having formula (IV) may be synthesized as described in: U.S. Pat. No. 4,968,697 or J. Med. Chem., 33, 1919-1924, (1990), which are incorporated by reference herein.

Another agonist compound that can be useful in the present invention is IB-MECA:

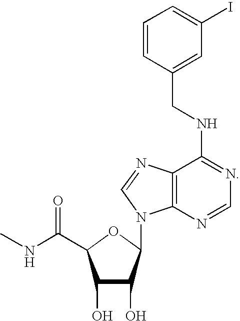

It will be appreciated by those skilled in the art that the compounds of formulas described herein, e.g., (I), (II), (III), and (IV), have more than one chiral center and may be isolated in optically active and racemic forms. In one embodiment, the riboside moiety of the compounds is derived from D-ribose, i.e., the 3′,4′-hydroxyl groups are alpha to the sugar ring and the 2′ and 5′ groups is beta (3R, 4S, 2R, 5S). When the two groups on the cyclohexyl group are in the 1- and 4-position, they are preferably trans. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, or enzymatic techniques, by synthesis from optically-active starting materials, by chiral synthesis, or by chromatographic separation using a chiral stationary phase) and how to determine adenosine agonist activity using the tests described herein, or using other similar tests which are well known in the art.

The preparation of the A_{2A} compounds described herein can be accomplished by a number of methods known to those of skill in the art including the patents and publications described previously. Additional examples of such methods include those described in U.S. Pat. Nos. 5,877,180; 6,232,297; 6,514,949; and, 7,214,665, and U.S. Patent Application Nos.: 2003/0186926; 2006/0217343; 2006/0040888; 2006/0030889; and, 2007/027073, which are incorporated herein by reference.

The following definitions are used, unless otherwise described.

Enteritis is intestinal inflammation.

Intestinal damage is disruption of the intestinal mucosa.

Diarrhea is defined as an increased frequency and/or decreased consistency of bowel movements. An example of diarrhea includes three or more unformed stools per day.

A_{2A} agonist refers to an agent that activates the Adenosine A_{2A} receptor with a Ki of <1 μM. An A_{2A} agonist may be selective for A_{2A} (e.g., at least 10, 50, or 100/1 over another adenosine receptor subtype/A₂A receptor). An A_{2A} agonist may also be cross reactive with other adenosine receptor subtypes (e.g., A₁, A_{2B}, and A₃). The A_{2A} agonist may activate other receptors with a greater or lesser affinity than the A_{2A} receptor.

“Treating” or “treatment” covers the treatment of a disease-state in a mammal, and includes: (a) preventing the disease-state from occurring in a mammal, in particular, when such mammal is predisposed to the disease-state, but has not yet been diagnosed as having it; (b) inhibiting the disease-state, e.g., arresting its development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

Halo is fluoro, chloro, bromo, or iodo. Alkyl, alkoxy, aralkyl, alkylaryl, etc. denote both straight and branched alkyl groups; but reference to an individual radical such as “propyl” embraces only the straight chain radical, a branched chain isomer such as “isopropyl” being specifically referred to. Aryl includes a phenyl radical or an ortho-fused bicyclic carbocyclic radical having about nine to ten ring atoms in which at least one ring is aromatic. Heteroaryl includes a radical attached via a ring carbon of a monocyclic aromatic ring containing five or six ring atoms consisting of carbon and one to four heteroatoms selected from non-peroxide oxygen, sulfur, and amine (—N(X)—), wherein X is absent or is hydrogen, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Specific and preferred values listed below for radicals, substituents, and ranges, are for illustration only; they do not exclude other defined values or other values within defined ranges for the radicals and substituents.

For example, (C₁-C₈)alkyl can be methyl, ethyl, propyl, isopropyl, butyl, iso-butyl, sec-butyl, pentyl, 3-pentyl, hexyl, heptyl or octyl. The term “cycloalkyl” includes bicycloalkyl (norbornyl, 2.2.2-bicyclooctyl, etc.) and tricycloalkyl (adamantyl, etc.), optionally comprising 1-2 N, O or S. Cycloalkyl also includes (cycloalkyl)alkyl. Thus, (C₃-C₈)cycloalkyl can be cyclopropyl, cyclobutyl, cyclopentyl, cyclohexyl and the like. (C₁-C₉)alkoxy can be methoxy, ethoxy, propoxy, isopropoxy, butoxy, iso-butoxy, sec-butoxy, pentoxy, 3-pentoxy, or hexyloxy; (C₂-C₈)alkenyl can be vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, or 5-hexenyl; (C₂-C₈)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₈)alkanoyl can be acetyl, propanoyl or butanoyl; halo(C₁-C₈)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₉)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₈)alkoxycarbonyl (CO₂R²) can be methoxycarbonyl, ethoxycarbonyl, propoxycarbonyl, isopropoxycarbonyl, butoxycarbonyl, pentoxycarbonyl, or hexyloxycarbonyl; (C₁-C₈)alkylthio can be methylthio, ethylthio, propylthio, isopropylthio, butylthio, isobutylthio, pentylthio, or hexylthio, (C₂-C₈)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyraxolyl, pyrrolyl, pyrazinyl, tetrazolyl, puridyl (or its N-oxide), thienyl, pyrimidinyl (or its N-oxide), indolyl, isoquinolyl (or its N-oxide) or quinolyl (or its N-oxide).

Aryl denotes a phenyl radical or an ortho-fused bicyclic carbocyclic radical having about nine to ten ring atoms in which at least one ring is aromatic. Heteroaryl denotes a radical of a monocyclic aromatic ring containing five or six ring atoms consisting of carbon and 1, 2, 3, or 4 heteroatoms selected from non-peroxide oxygen, sulfur, and amine (—N(Y)—) wherein Y is absent or is hydrogen, O, (C₁-C₈)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

The term “heterocycle” generally represents a non aromatic heterocyclic group, having from 3 to about 10 ring atoms, which can be saturated or partially unsaturated, containing at least one heteroatom (e.g., 1, 2, or 3), where the heteroatom is oxygen, nitrogen, or sulfur. Exemplary, “heterocycle” groups include monocyclic, bicyclic, or tricyclic groups containing one or more heteroatoms selected from oxygen, nitrogen, and sulfur. A “heterocycle” group also can include one or more oxo groups (═O) attached to a ring atom. Non-limiting examples of heterocycle groups include 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuelidine, thiomorpholine, and the like.

The term “alkylene” refers to a divalent straight or branched hydrocarbon chain (e.g. ethylene —CH₂CH₂—).

The term “aryl(C₁-C₉)alkylene” includes benzyl, phenethyl, 3-phenylpropyl, naphthylmethyl and the like.

The carbon atom content of various hydrocarbon-containing moieties is indicated by a prefix designating the minimum and maximum number of carbon atoms in the moiety, e.g., the prefix Cᵢ-Cⱼ indicates a moiety of the integer “i” to the integer “j” carbon atoms, inclusive. Thus, for example, (C₁-C₈)alkyl refers to alkyl of one to eight carbon atoms, inclusive. The compounds herein are generally named according to the IUPAC or CAS nomenclature system. Abbreviations which are well known to one of ordinary skill in the art may be used (e.g., “Ph” for phenyl, “Me” for methyl, “Et” for ethyl, “h” for hour or hours and “rt” for room temperature).

The phrase “stable glutamine” or “stable glutamine derivative” generally relates to a glutamine with a lower reactivity than glutamine with respect to its propensity toward cyclization to pyroglutamate. Typically, a stable glutamine has its amine group acylated or sulfonylated. Stable glutamine derivatives can be prepared by coupling glutamine with one or more additional amino acids to provide oligopeptides, or with glucose, or both, or acylating glutamine with a carboxylic acid having 2 to 6 carbon atoms, to provide a compound which is stable to degradation under acidic environments. Any naturally occurring amino acid may be used as the additional amino acid coupled to the glutamine, including alanine or glutamine, alone or in combination. Examples of the number of total amino acid groups present include from 2 to 5 (formed from coupling from 1 to 4 amino acids with glutamine), which includes dipeptides and tripeptides. Specific examples include alanyl-glutamine, alanyl-glutaminyl glutamine and gamma-glutamyl glutamine. Stable glutamines can be prepared using known methodology (e.g., conventional peptide coupling reactions) as described in U.S. Pat. No. 5,561,111, which is incorporated herein by reference.

The term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

The term “pharmaceutically acceptable carrier” includes any of the standard pharmaceutical carriers known in the art, such as a phosphate buffered saline solution, hydroxypropyl beta-cyclodextrins (HO-propyl beta cyclodextrins), water, emulsions such as an oil/water or water/oil emulsion, and various types of wetting agents. The term also includes any of the agents approved by a regulatory agency of the U.S. Federal government or listed in the US Pharmacopeia for use in animals, including humans.

The term “sample,” refers preferably to a biological sample from a subject, including, but not limited to, normal tissue samples, diseased tissue samples, biopsies, blood, saliva, feces, semen, tears, and urine. A sample can also be any other source of material obtained from a subject, which contains cells, tissues, or fluid of interest. A sample can also be obtained from cell or tissue culture.

The term “standard,” as used herein, refers to something used for comparison. For example, it can be a known standard agent or compound which is administered or added to a control sample and used for comparing results when measuring said compound in a test sample. Standard can also refer to an “internal standard”, such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals, preferably a human. A “subject” of diagnosis or treatment is a mammal, including a human.

A “therapeutic” treatment is a treatment administered to a subject who exhibits signs of pathology for the purpose of diminishing or eliminating those signs. A “therapeutically effective amount” of a compound is that amount of compound which is sufficient to provide a beneficial effect to the subject to which the compound is administered.

The term “effective amount” means an amount sufficient to produce a selected effect.

The “term “pharmaceutically-acceptable salt” refers to salts which retain the biological effectiveness and properties of the compounds of the present invention and which are not biologically or otherwise undesirable. In many cases, the compounds of the present invention are capable of forming acid and/or base salts by virtue of the presence of amino and/or carboxyl groups or groups similar thereto.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Exemplary inorganic salts that may also be formed, include hydrochloride, sulfate, nitrate, bicarbonate, and carbonate salts.

Pharmaceutically acceptable salts may be obtained using standard procedures well known in the art, for example by reacting a sufficiently basic compound such as an amine with an acid affording a physiologically acceptable anion. Alkali metal (for example, sodium, potassium or lithium) or alkaline earth metal (for example calcium) salts of carboxylic acids can also be made.

Dosages and Formulations

The A_{2A} compounds can conveniently be administered in a pharmaceutical composition containing the compound in combination with an excipient. The stable glutamine derivatives can conveniently be administered in a pharmaceutical composition containing the compound in combination with an excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15th Ed., 1975). The compounds and compositions can be administered parenterally (for example, by intravenous, intraperitoneal or intramuscular injection), topically, orally, and/or rectally.

For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of compounds having formula (I), (III) or (IV) can be determined by comparing their in vitro activity, and in vivo activity in animal models. Methods for the extrapolation of effective dosages in mice, and other animals, to humans are known to the art; for example, see U.S. Pat. No. 4,938,949, which is incorporated herein by reference.

The compound is conveniently administered in unit dosage form; for example, containing about 0.05 mg to about 500 mg, conveniently about 0.1 mg to about 250 mg, most conveniently, about 1 mg to about 150 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compositions can conveniently be administered orally, sublingually, transdermally, or parenterally at dose levels of about 0.01 to about 150 μg/kg, preferably about 0.1 to about 50 μg/kg, and more preferably about 0.1 to about 10 μg/kg of mammal body weight.

For parenteral administration the compounds are presented in aqueous solution in a concentration of from about 0.1 to about 10%, more preferably about 0.1 to about 7%. The solution may contain other ingredients, such as emulsifiers, antioxidants or buffers.

The invention is now described with reference to the following Examples and Embodiments. Without further description, it is believed that one of ordinary skill in the art can, using the preceding description and the following illustrative examples, make and utilize the disclosed compositions. The following working examples therefore, are provided for the purpose of illustration only and specifically point out the preferred embodiments, and are not to be construed as limiting in any way the remainder of the disclosure. Therefore, the examples should be construed to encompass any and all variations that become evident as a result of the teaching of the specification.

### EXAMPLES

ATL 313 is: 4-{3-[6-Amino-9-(5-cyclopropylcarbamoyl-3,4-dihydroxy-tetrahydro-furan-2-yl)-9H-purin-2-yl]-prop-2-ynyl}-piperidine-1-carboxylic acid methyl ester.

ATL 146e is: 4-{3-[6-Amino-9-(5-ethylcarbamoyl-3,4-dihydroxy-tetrahydro-furan-2-yl)-9H-purin-2-yl]-prop-2-ynyl}-cyclohexanecarboxylic acid methyl ester.

JMR 193 is: Acetic acid 4-{3-[6-amino-9-(5-ethylcarbamoyl-3,4-dihydroxy-tetrahydro-furan-2-yl)-9H-purin-2-yl]-prop-2-ynyl}-cyclohexylmethyl ester.

ATL 370 is: 4-{3-[6-Amino-9-(5-cyclopropylcarbamoyl-3,4-dihydroxy-tetrahydro-furan-2-yl)-9H-purin-2-yl]-prop-2-ynyl}-piperidine-1-carboxylic acid 4-chloro-phenyl ester.

### Example 1

The ability of a disclosed compound to treat infections caused by C. difficile may be determined using pharmacological models which are well known to the art, or using those described in “Effect of Novel A_{2A} Adenosine Receptor Agonist ATL 313 on Clostridium difficile Toxin A-Induced Murine Ileal Enteritis” Infection and Immunity, May 2006, p. 2606-2612, Vol. 74, No. 5, which is incorporated herein by reference.

Materials And Methods

Animals

For this study, 174 male Swiss mice, 25-30 g body weight, from the animal colony of the Federal University of Ceara were used. The animals received water and food ad libitum.

Drugs and Toxins

The following drugs were used: purified toxin A from Clostridium difficile (strain #10463; molecular weight 308 kDa), kindly provided through collaboration with Dr. David Lyerly, Tech Lab, Blacksburg, Va.; 4-{3-[6-amino-9-(5-cyclopropylcarbamoyl-3,4-dihydroxytetrahydrofuran-2-yl)-9H-purin-2-yl]prop-2-ynyl}piperidine-1-carboxylic acid methyl ester (ATL 313) (kindly provided by Adenosine Therapeutics, LLC, USA); all these substances were diluted in PBS pH 7.4. 4-[2-(7-Amino-2-furan-2-yl-[1,2,4]triazolo[1,5-a][1,3,5]triazin-5-ylamino)-ethyl]-phenol (ZM241385) was a gift from Simon Poucher, Astra-Zeneca Pharmaceuticals (Cheshire, UK).

Induction of Intestinal Inflammation

Mice were fasted overnight, but allowed access to water, and then anesthetized with ketamine and xylazine (60 and 5 mg/Kg intramuscularly, respectively). Through a midline laparotomy, one 4-cm ileal loop was ligated and injected with either 0.1 ml of PBS pH 7.4 (control) or buffer containing TxA (1-10 μg). The abdomen was closed, and the animals were allowed to regain consciousness. Three hours after administration of TxA, mice were sacrificed and the intestinal loops were removed. The loop length, weight, and fluid volume were recorded. A portion of the loop was frozen at −70° C. for measurement of myeloperoxidase (MPO) and ADA activities and TNF-α concentration. The remaining tissue was fixed in 10% formalin and embedded in paraffin, and sections were stained with hematoxylin and eosin for histological grading of ileal inflammation. Some mice were injected with ATL 313 (0.05, 0.05 or 5 mM final concentration) immediately followed by PBS or TxA (5 μg); another group was injected with ZM241385 (5 nM), the selective A_{2A} AR antagonist, immediately followed by PBS or ATL 313 (5 nM)+TxA (5 μg) in the ileal loop. In another experiment, TxA (5 μg) was injected into the ileal loop, the abdomen was closed, and, 30 min later, the abdomen was reopened and ATL 313 (5 nM) administered.

Histology

The severity of inflammation was scored in coded slides by a pathologist on a scale of 1 (mild) to 3 (severe) for epithelial damage, edema, and neutrophil infiltration.

Cell Death

Intestinal sections were also processed for terminal deoxynucleotidyl transferase-mediated dUTP-biotin nick end-labeling (TUNEL method) using ApopTag® Plus Peroxidase in Situ Detection Kit (Serologicals Corporation, Norcross, Ga.) for analysis of apoptosis or necrosis. Briefly, paraffin-embedded sections were hydrated and incubated with 20 μg/mL of proteinase K (Sigma, N.Y., USA) for 15 min at room temperature. Endogenous peroxidase was blocked by treating with 3% (w/v) hydrogen peroxide, in PBS for 5 min at room temperature. After washing, sections were incubated in a humidified chamber at 37° C. for 1 hr with TdT buffer, containing TdT enzyme and reaction buffer. Specimens were incubated for 10 min at room temperature with a stop/wash buffer and then incubated in a humidified chamber for 30 min with anti-digoxigenin peroxidase conjugate at room temperature. After a series of washes in phosphate buffered saline, the slides were covered with peroxidase substrate to develop color and then wash in 3 changes of dH₂O and counterstained in 0.5% (v/v) methyl green for 10 min at room temperature.

MPO Assay

The extent of neutrophil accumulation in ileal tissue was estimated by measuring MPO activity assay. Briefly, 50-100 mg of ileal tissue was homogenized in 1 mL of Hexadecyltrimethylammonium bromide (HTAB) buffer for each 50 mg of tissue. Then, the homogenate was centrifuged at 4,000×g for 7 min at 4° C. MPO activity in the resuspended pellet was assayed by measuring the change in absorbance at 450 nm using o-dianisidine dihydrochloride and 1% hydrogen peroxide. The results were reported as MPO units/mg of tissue. A unit of MPO activity was defined as that converting 1 μmol of hydrogen peroxide to water in 1 min at 22° C.

Quantification of TNF-α by ELISA

Ileal tissue was harvested from animals in order to measure TNF-α concentration by enzyme-linked immunosorbent assay (ELISA). The results are expressed as pg/mL of TNF-α.

ADA Activity

Tissue samples and their contents were collected from PBS and TxA injected mice. The tissues were homogenized in 8 volumes of cold phosphate buffer (50 mmols/L, pH 7.2). This preparation and the ileum contents were centrifuged at 10,000×g in a refrigerated centrifuge at 5-8° C., for 30 min. The sediments were discarded and supernatants assayed for ADA activity and protein content.

The ADA assay is based on the measurement of ammonia produced during the deamination of adenosine by the method of Giusti (1974. Adenosine deaminase, p. 1092-1099. In H. U. Bergmeyer (Ed.), Methods of Enzymatic Analysis, vol. 2. Academic Press Inc., New York), with slight modifications. In brief, to a final volume of 220 μL, 200 μL of adenosine (21 mM) in phosphate buffer (50 mM, pH7.2) were added to the sample (20 μL; supernatant of tissue homogenate or ileum secretion). A control tube (200 μL of adenosine 21 mM), a standard tube (200 μL of ammonium sulphate 75 μM in phosphate buffer) and a blank tube (200 μL of phosphate buffer) were also produced. One hour after incubation at 37° C. the reactions were stopped with 600 μL of phenol/potassium nitroprussiate (106 mM/101.7 mM) and 20 μL of sample were added to control, standard and blank tubes. Following the addition of 600 μl of sodium hypochloride (11 mM) in 125 mM NaOH all tubes were again incubated at 37° C. for 30 min and read at 628 nm. The enzyme activity in the tissue and in the ileum contents was expressed as μmols of ammonium formed/mg of protein/hour and as μmols of ammonium formed/hour, respectively.

Statistics

Results are reported as means ±SEM or as median values and range, where appropriate. Univariate ANOVA followed by Bonferroni's test was used to compare means, and the Kruskal-Wallis followed by Dunn's test was used to compare medians. A probability value of P<0.05 was considered to indicate significant differences.

Results

Effect of Clostridium difficile Toxin A (TxA) in Murine Ileal Loops.

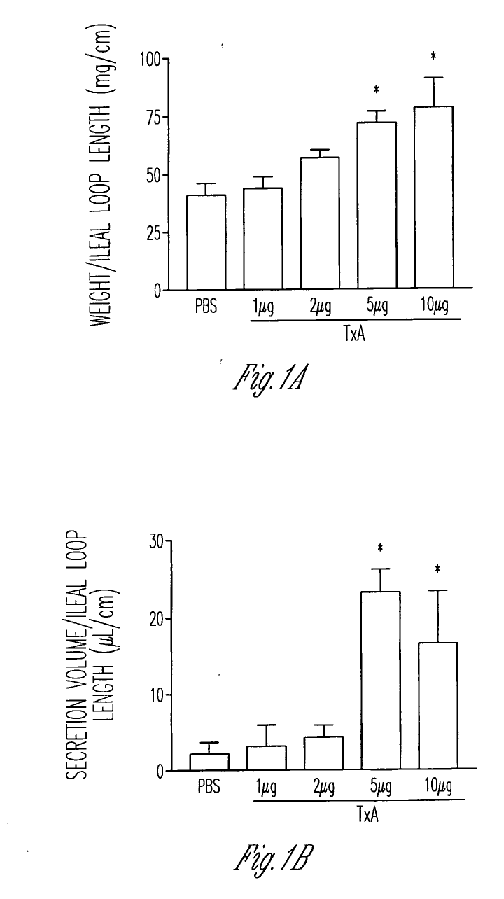

To evaluate the inflammatory and secretory effects of TxA in murine ileal loops, we observed enteritis in response to graded amounts of TxA exposure (1 μg, 2 μg, 51 g and 10 μg) measuring both loop weight and accumulation of fluid in the intestinal lumen as endpoints. There was a trend toward higher ileal weight/length and volume/length ratios at 1-2 μg of TxA that reached statistical significance (P<0.05) at 5 μg and 10 μg, compared to the PBS control (FIGS. 1A and 1B).

Effect of ATL 313 on Murine Ileal Loops Injected with Clostridium difficile Toxin A (TxA).

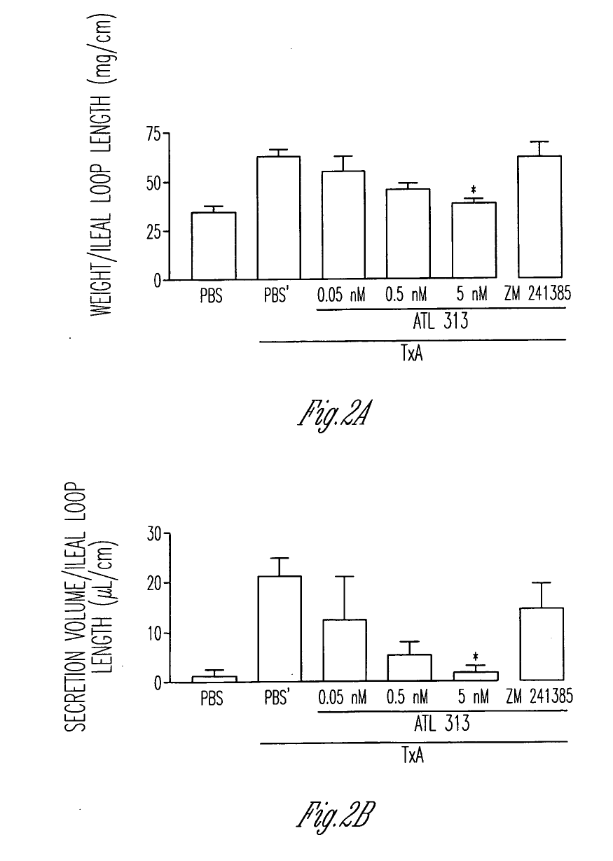

Treatment with the A_{2A} AR agonist, ATL 313 (5 nM), significantly (P<0.05) reduced the TxA (5 μg)-induced increase in weight/ileal loop length and secretion volume/ileal loop length ratios. ATL 313 (5 nM) alone did not alter weight/ileal loop length and secretion volume/ileal loop length ratios in the absence of TxA (FIGS. 2A and 2B).

At an equimolar concentration (5 nM), the A_{2A} AR antagonist, ZM241385, significantly reversed the protective effect of ATL 313, restoring both weight/ileal loop length and secretion volume/ileal loop length ratios to levels comparable to TxA challenge alone (FIGS. 2A and 2B). ZM241385 alone did not increase weight/ileal loop length (ZM241385=19.9±0.9 vs. PBS=34.5±3.2 mg/cm) and secretion volume/ileal loop length (ZM241385=0.0±0.0 vs. PBS=1.25±1.25 μL/cm) ratios.

The treatment with ATL313 30 min after injection of TxA also significantly (P<0.05) reduced the TxA (5 μg)-induced increase in weight/ileal loop length ratio (TxA=39.9±3.5 vs. ATL 313=28.3±1.4 mg/cm).

Effect of ATL 313 on Clostridium difficile Toxin A (T×A)-Induced Histological Alterations and Cell Death.

Histological analysis demonstrated that TxA (5 μg/loop) induces intense mucosal disruption, hemorrhage, edema, and inflammatory cell infiltration, resulting in a median injury score of 3 and a range of 2-3. TxA also caused a large amount of mucosal cell death in mouse ileal loops, compared to PBS which received median score 0(0-0). The group treated with ATL 313 was significantly (P<0.05) protected from the disruptive effects of TxA receiving a median score of 1(0-2), and exhibited a reduced number for cell death similar to the level of PBS control. The A_{2A} AR antagonist (ZM241385) blocked the protective effect of ATL 313, with damage (median score: 2.5 and a range of 2-3 similar to the group challenged with TxA alone. Of importance, neither ATL 313 nor ZM241385 alone induced any histological evidence of injury receiving a median score of 0(0-0).

Treatment with ATL 313 30 min after injection of TxA also significantly (P<0.05) reduced the TxA (5 μg)-induced histological evidence of injury resulting in a score of 1(0-2).

Effect of ATL 313 on Clostridium difficile Toxin A-Induced MPO Activity.

MPO is an enzyme present in the azurophil granules of neutrophils, and its presence in tissues has been used as an index of neutrophil infiltration. TxA (5 μg/loop) caused a statistically significant increase (P<0.05) in MPO activity in ileal tissue, compared to the loops from the control group, injected with only PBS (TxA=12.6±2.6 vs. PBS=1.6±0.6 U/mg).

The group treated with the A_{2A} AR agonist, ATL 313 (5 nM), and then challenged with TxA had markedly reduced MPO activity (P<0.05; ATL 313+TxA=1.0±0.6 vs. TxA=12.6±2.6 U/mg). MPO activity in ileal tissue collected from animals injected with ATL 313 plus TxA was comparable to that observed in the PBS control animals. ATL 313 (5 nM) alone did not increase MPO activity compared to PBS (ATL 313=2.1±0.2 vs. PBS=1.6±0.6 U/mg).

Effect of ATL313 on Clostridium difficile Toxin A-Induced TNF-α Production.

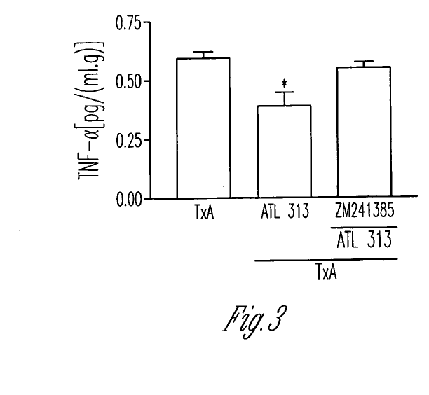

The injection of TxA (5 μg/loop) into mouse ligated ileal loops significantly increased TNF-α production within the ileal tissue (P<0.05), compared with the control group challenged only with PBS. Treatment with ATL 313 significantly reduced (P<0.05) TNF-α production in ileal tissue, compared to loops injected with only TxA. The selective A_{2A} AR antagonist, ZM241385, blocked ATL313 action, promoting a raise in TNF-α expression, which reached levels similar to those seen in the group injected with only TxA (FIG. 3).

Effect of ATL 313 on Clostridium difficile Toxin A-Induced ADA Activity.

The injection of TxA (5 μg/loop) into mouse ligated ileal loops significantly increased ADA activity within the ileal tissue (P<0.05) and ileal secretion, compared with the control group challenged only with PBS (FIGS. 4A and 4B).

Treatment with ATL 313, the A_{2A} AR agonist, significantly reduced (P<0.05) ADA activity in ileal tissue and ileal secretion in TxA-challenged animals (FIGS. 4A and 4B).

### Example 2

Materials and Methods: New Zealand white rabbits weighing about 2 kg are fasted overnight. Under anesthesia with ketamine (60-80 mg/kg) and xylazine (5-10 mg/kg) administered intramuscularly, a midline abdominal incision is performed to expose the small bowel. After the ileum is flushed, four-cm loops are ligated using double ties with a 1 cm interval between loops. Each of the control loops is injected intraluminally with 1 mL solution of PBS with either C. difficile toxin A (toxin A) (Techlab, Blacksburg, Va.) at 10 μg/mL. Loops from another set of rabbits are treated with the adenosine A_{2A} agonists, JMR 193 (ATL 193) (10 nM, 100 nM, or 1 uM doses) or ATL 146e (10 mM, 100 nM, 1 uM doses) immediately before the enterotoxin is administered. In addition a rabbit is treated with A_{2A} agonist, ATL 313 (100 nM), alone and another rabbit treated with ATL 313 with alanyl-glutamine (25 mM). After 5 hours of incubation, the ligated small intestinal loops are removed. The length of each ligated ileal segment is measured and intraluminal fluid was quantified. Volume to length ratio, V/L (mL/cm), per loop is calculated. Samples of intestinal stained with hematoxylin-eosin and graded from 0 (none) to 4 (worst) based on the degree of mucosal disruption, increase in cellularity and intensity of vascular congestion.

For other types of colitides, the same rabbit ileal loop model may be utilized to check for the effect of the adenosine receptor analogues on other toxin- or pathogen-induced enterocolitis.

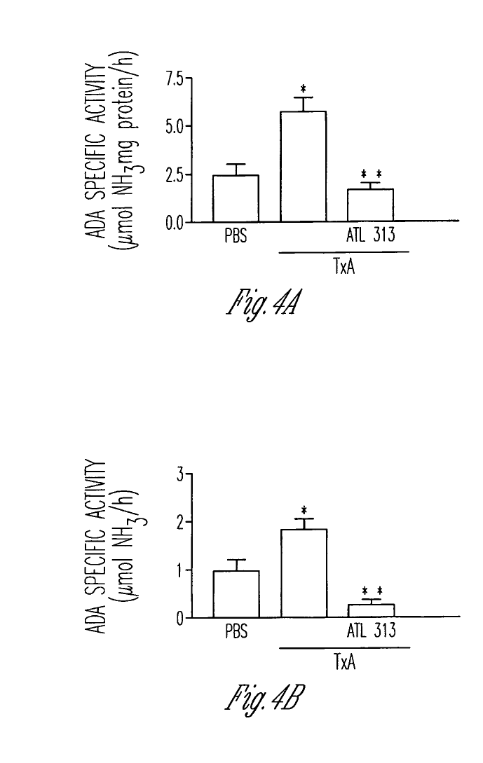

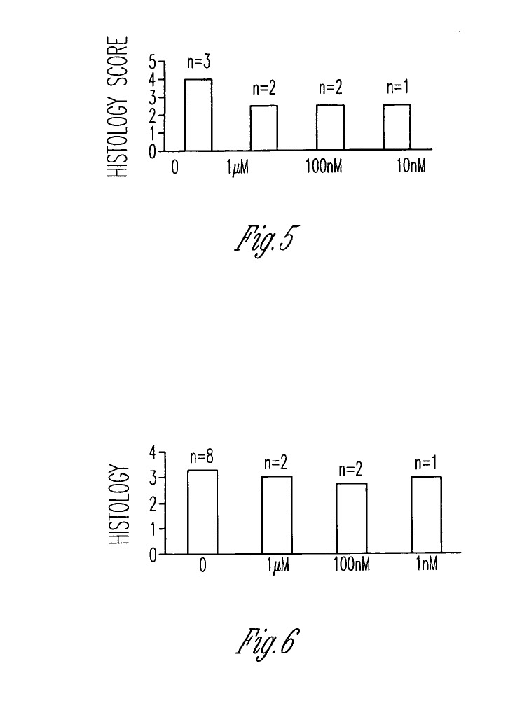

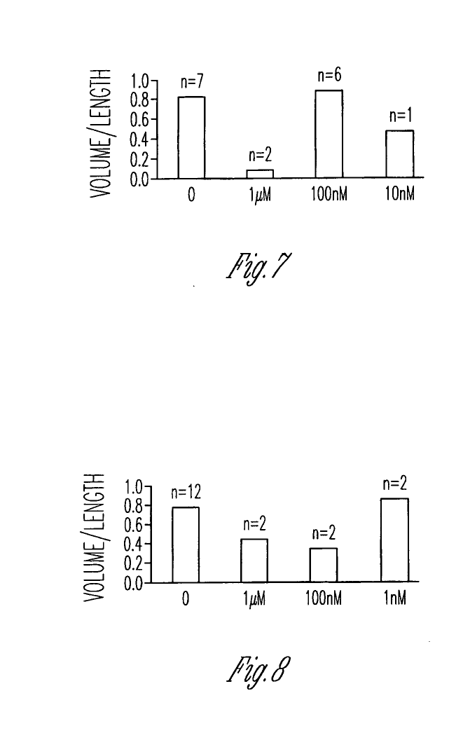

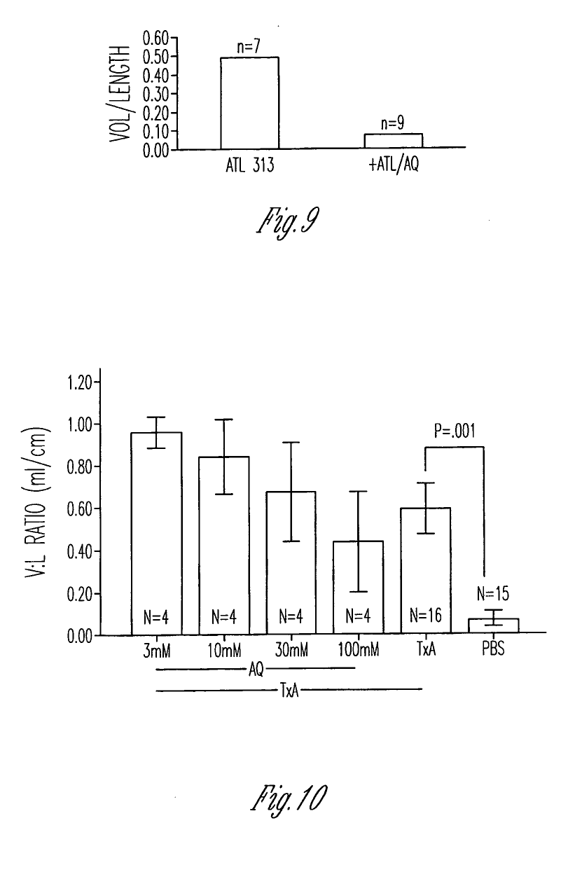

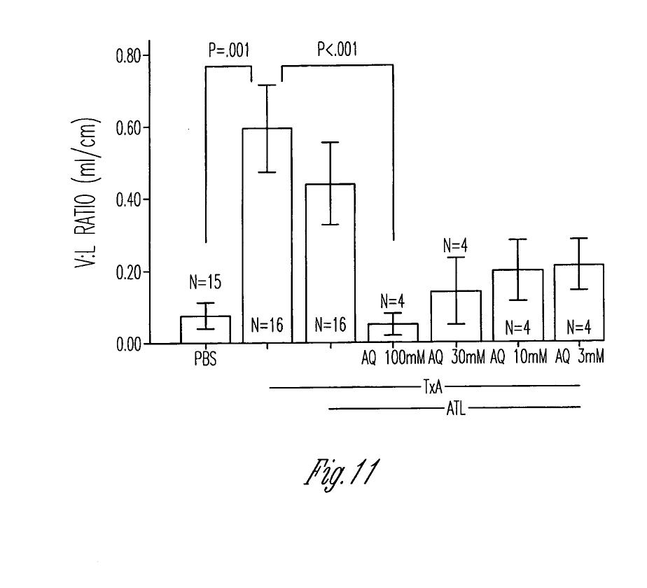

Results: As shown in FIGS. 5 and 6, inflammation induced by toxin A was reduced by pretreatment with A_{2A} agonists, JMR 193 or ATL 146e. Secretion, however, was not consistently ameliorated, see FIGS. 7 and 8. As shown in FIG. 9, the combination of alanyl-glutamine with A_{2A} agonist, ATL 313, dramatically improved secretion in addition to the improved inflammation.

Conclusion: the A_{2A} agonists are useful to treat inflammatory and secretory diarrhea. Having an effect on C. difficile toxin A-induced ileal loops, the A_{2A} agonists may be used also for other toxin- or pathogen-induced inflammatory diarrhea such as Campylobacter, Shigella, Salmonella, Yersinia, enterohemorragic E. coli, and others. Moreover, secretory diarrhea caused by Cryptosporidium, viruses, enteropathogenic and enterotoxigenic E. coli and others may benefit from the antisecretory effect of A_{2B} antagonists. Addition of alanyl-glutamine improves the effect of A_{2A} agonists by the additional antisecretory effect and repair of the toxin A-induced mucosal injury.

### Example 3

Rabbit Ileal Loops: The animal experiment protocol was approved by the Animal Care and Use Committee at the University of Virginia. Eight New Zealand white rabbits weighing between 2.5-3 kg were used for this experiment. Each rabbit was fasted overnight. On the day of the experiment, the rabbits were anesthetized intramuscularly using a ketamine and xylazine solution in a 2:1 ratio. An intravenous line was placed in the ear vein of each rabbit for administration of the adenosine analog, ATL 370 (Adenosine Therapeutics) or a phosphate buffered saline placebo (PBS). Once anesthetized, a midline abdominal incision was made and the ileum was exposed. A measured amount of ileum was injected with 5 mL of PBS to flush any remaining fecal material. After the ileum was flushed, 10 loops of 2.5-5 cm each were ligated using double ties, with 0.5-1 cm of space between each loop.

Once the bowel was exposed and ligated, each rabbit received a 250 μL IV injection of PBS (Toxin A and Toxin A/Alanyl glutamine groups) or 1.5 ug/250 μL of ATL 370 (Toxin A/Adenosine and Toxin A/Adenosine/Alanyl-glutamine groups). An additional dose of either PBS or ATL 370 was administered 2 hours after the first dose for a total of 2 doses. Each dose was followed by a 250-500 μL heparin saline injection to prevent the IV from clotting.

A total of 79 loops were studied, of which 15 loops from 8 animals served as controls. Each of the control loops were injected intraluminally with 1 mL of a sterile PBS solution. The remaining 64 loops were injected intraluminally with 20 μg/500 μL of Toxin A (TechLab) and combined with either 500 μL PBS (Toxin A and Toxin A/Adenosine groups) or 500 μL of a 10 mM, 30 mM, 10 mM, or 3 mM alanyl-glutamine solution (Toxin A/Alanyl-glutamine and Toxin A/Adenosine/Alanyl-glutamine groups). The ileal loops were replaced intraperitoneally and the abdomimal incision was sutured. Sedation continued throughout the procedure and were euthanized after 4 hours.

Measurement of intestinal secretion: After 4 hours of incubation, the animals were euthanized and the ligated loops were immediately removed. The length of each loop was measured and incised and the intraluminal secretion was quantified. The volume to length ratio (V:L) was measured as milliliters per centimeter per loop. The gross description of intestinal fluid (serous, serosanguinous, hemorrhagic or purulent) was documented.

Results: TxA induced secretion: A total of 16 loops were injected with toxin A only. Toxin A treated loops had a significantly elevated V:L ratio as compared to PBS loops (0.60 mL/cm, n=16 vs 0.08 mL/cm, n=15; p=0.001). The majority (94%, 14/16) of toxin A treated loops were hemorrhagic, purulent, or a combination of the two. The V:L ratios of the toxin A group as compared to the combinations of toxin A mixed with alanyl-glutamine at 100 mM, 30 mM, 10 mM, or 3 mM (0.60 mL/cm vs. 0.44 mL/cm, 0.67 mL/cm, 0.84 mL/cm, and 0.96 mL/cm respectively) were not significantly different, but showed a trend towards increasing V:L ratios with decreasing doses of alanyl-glutamine. The majority (73%, 11/15) of toxin A/alanyl-glutamine loops were hemorrhagic, purulent, or a combination of the two. Rabbits treated with TV doses of adenosine and intraluminal injections of Toxin A only had a lower V:L ratio as compared to Toxin A only groups, but did not reach statistical significance (0.44 mL/cm, n=8 vs 0.60 mL/cm, n=16, p=0.35). The majority (63%, 10/16) were either hemorrhagic or purulent. Rabbits treated with a combination of adenosine IV, toxin A, and 100 mM of alanyl-glutamine showed a significantly decreased V:L ratio as compared with the toxin A only group (0.05 mL/cm, n=4 vs. 0.60, n=16; p<0.001). A trend towards decreased V:L ratios was demonstrated with 30 mM, 10 mM, and 3 mM doses of alanyl-glutamine (0.14 mL/cm, 0.20 mL/cm, and 0.21 mL/cm respectively). 44% (7/16) were purulent and only one was hemorrhagic.

All patents, patent applications and literature cited in the specification are hereby incorporated by reference in their entirety. In the case of any inconsistencies, the present disclosure, including any definitions therein will prevail. The invention has been described with reference to various specific and preferred embodiments and techniques. However, it should be understood that many variations and modifications may be made while remaining within invention.

What is claimed is:

1. A method for treating a C. difficile or C. difficile toxin A induced condition comprising administering to a patient in need thereof an effective amount of an A_{2A} adenosine receptor agonist, wherein the condition is selected from intestinal damage, enteritis, diarrhea, and a combination thereof.

2. The method of claim 1, further comprising administering, in combination with the A_{2A} adenosine receptor agonist, an effective amount of a stable glutamine derivative.

3. The method of claim 2, wherein the stable glutamine derivative is alanyl-glutamine.

4. The method of claim 1, wherein the agonist comprises a substituted 6-amino-9-(tetrahydrofuran-2′-yl)purine, or a pharmaceutically acceptable salt thereof.

5. The method of claim 1 wherein the agonist comprises a 6-amino-9-(3′,4′-dihydroxy-tetrahydrofuran-2′-yl)purine substituted at the 3- and 5′-positions, or a pharmaceutically acceptable salt thereof.

6. The method of claim 1 wherein the agonist comprises a 5-[6-amino-2-(3-piperidin-4-yl-prop-1-ynyl)-purin-9-yl]-3,4-dihydroxy-tetrahydro-furan-2-carboxylic acid cyclopropylamide, substituted on the piperidine nitrogen, or a pharmaceutically acceptable salt thereof.

7. The method of claim 1 wherein the agonist comprises a 5-[6-amino-2-(3-piperidin-4-yl-prop-1-ynyl)-purin-9-yl]-3,4-dihydroxy-tetrahydro-furan-2-carboxylic acid ethylamide, substituted on the piperidine nitrogen, or a pharmaceutically acceptable salt thereof.

8. The method of claim 1 wherein the agonist comprises a 4-{3-[6-amino-9-(5-cyclopropylcarbamoyl-3,4-dihydroxy-tetrahydro-furan-2-yl)-9H-purin-2-yl]-prop-2-ynyl}-piperidine-1-carboxylic acid ester or a pharmaceutically acceptable salt thereof.

9. The method of claim 1 wherein the agonist comprises a 4-{3-[6-amino-9-(5-ethylcarbamoyl-3,4-dihydroxy-tetrahydro-furan-2-yl)-9H-purin-2-yl]-prop-2-ynyl}-piperidine-1-carboxylic acid ester or a pharmaceutically acceptable salt thereof.

10. The method of claim 1, wherein the A_{2A} adenosine receptor agonist is a compound of formula I or a stereoisomer or pharmaceutically acceptable salt thereof: 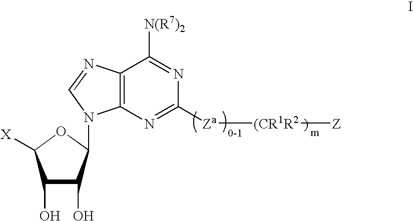
wherein
Z^{a} is C≡C, O, NH, or NHN═CR^{3a};
Z is CR³R⁴R⁵ or NR⁴R⁵;
each R¹ is independently hydrogen, halo, —OR^{a}, —SR^{a}, (C₁-C₉)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, or —N═NR^{b};
each R² is independently hydrogen, halo, (C₁-C₈)alkyl, (C₃-C₈)cycloalkyl, heterocycle, heterocycle(C₁-C₈)alkylene-, aryl, aryl(C₁-C₈)alkylene-, heteroaryl, or heteroaryl(C₁-C₈)alkylene-;
alternatively, R¹ and R² and the atom to which they are attached is C═O, C═S or C═NR^{d}, R⁴ and R⁵ are independently H or (C₁-C₈)alkyl;
alternatively, R⁴ and R⁵ together with the atom to which they are attached form a saturated, partially unsaturated, or aromatic ring that is mono-, bi- or polycyclic and has 3, 4, 5, 6, 7, 8, 9 or 10 ring atoms optionally having 1, 2, 3, or 4 heteroatoms selected from non-peroxide oxy (—O—), thio (—S—), sulfinyl (—SO—), sulfonyl (—S(O)₂—) or amine (—NR^{b}—) in the ring;
wherein R⁴ and R⁵ are independently substituted with 0-3 R⁶ groups or any ring comprising R⁴ and R⁵ is substituted with from 0 to 6 R⁶ groups;
each R⁶ is independently hydrogen, halo, —OR^{a}, —SR^{a}, (C₁-C₉)alkyl, cyano, nitro, trifluoromethyl, trifluoromethoxy, (C₁-C₈)cycloalkyl, (C₆-C₁₂)bicycloalkyl, heterocycle, heterocycle (C₁-C₈)alkylene-, aryl, aryl (C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, —NNR^{b}, or two R⁶ groups and the atom to which they are attached is C═O, C═S; or two R⁶ groups together with the atom or atoms to which they are attached can form a carbocyclic or heterocyclic ring comprising from 1-6 carbon atoms and 1, 2, 3, or 4 heteroatoms selected from non-peroxide oxy (—O—), thio (—S—), sulfinyl (—SO—), sulfonyl (—S(O)₂—) or amine (—NR^{b}—) in the ring;
R³ is hydrogen, halo, —OR^{a}, —SR^{a}, (C₁-C₈)alkyl, cyano, nitro, trifluoromethyl, trifluoromethoxy, (C₃-C₈)cycloalkyl, heterocycle, heterocycle(C₁-C₈)alkylene-, aryl, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)—, R^{a}S(═O)₂—, —NNR^{b}; or if the ring formed from CR⁴R⁵ is aryl or heteroaryl or partially unsaturated then R³ can be absent;
R^{3a} is hydrogen, (C₁-C₈)alkyl, or aryl;
each R⁷ is independently hydrogen, (C₁-C₈)alkyl, (C₃-C₈)cycloalkyl, aryl, aryl(C₁-C₈)alkylene, heteroaryl, or heteroaryl(C₁-C₈)alkylene-;
X is —CH₂OR^{a}, —CO₂R^{a}, —CH₂OC(O)R^{a}, C(O)NR^{b}R^{c}, —CH₂SR^{a}, —C(S)OR^{a}, —CH₂OC(S)R^{a}, —C(S)NR^{b}R^{c}, or —CH₂N(R^{b})(R^{c});
alternatively, X is an aromatic ring of the formula: 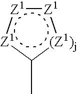
each Z¹ is non-peroxide oxy (—O—), S(O)₀₋₂, —C(R⁸)—, or amine (—NR⁸—), provided that at least one Z¹ is non-peroxide oxy (—O—), thio (—S—), sulfinyl (—SO—), sulfonyl (—S(O)₂—) or amine (—NR⁸—);
each R⁸ is independently hydrogen, (C₁-C₈)alkyl, (C₁-C₈)alkenyl, (C₃-C₈)cycloalkyl, (C₃-C₈)cycloalkyl(C₁-C₈)alkylene, (C₃-C₈)cycloalkenyl, (C₃-C₈)cycloalkenyl(C₁-C₈)alkylene, aryl, aryl(C₁-C₈)alkylene, heteroaryl, or heteroaryl(C₁-C₈)alkylene, wherein any of the alkyl or alkenyl groups of R⁸ are optionally interrupted by —O—, —S—, or —N(R)—;
wherein any of the alkyl, cycloalkyl, heterocycle, aryl, or heteroaryl, groups of R¹, R², R³, R^{3a}, R⁶, R⁷ and R⁸ is optionally substituted on carbon with one or more (e.g. 1, 2, 3, or 4) substituents selected from the group consisting of halo, —OR^{a}, —SR^{a}, (C₁-C₈)alkyl, cyano, nitro, trifluoromethyl, trifluoromethoxy, (C₃-C₈)cycloalkyl, (C₆-C₁₂)bicycloalkyl, heterocycle, heterocycle(C₁-C₈)alkylene-, aryl, aryloxy, aryl(C₁-C₈)alkylene-, heteroaryl, heteroaryl(C₁-C₈)alkylene-, —CO₂R^{a}, R^{a}C(═O)O—, R^{a}C(═O)—, —OCO₂R^{a}, R^{b}R^{c}NC(═O)O—, R^{a}OC(═O)N(R^{b})—, R^{b}R^{c}N—, R^{b}R^{c}NC(═O)—, R^{a}C(═O)N(R^{b})—, R^{b}R^{c}NC(═O)N(R^{b})—, R^{b}R^{c}NC(═S)N(R^{b})—, —OPO₃R^{a}, R^{a}OC(═S)—, R^{a}C(═S)—, —SSR^{a}, R^{a}S(═O)ₚ—, R^{b}R^{c}NS(O)ₚ—, and —N═NR^{b};
wherein any (C₁-C₈)alkyl, (C₃-C₈)cycloalkyl, (C₆-C₁₂)bicycloalkyl, (C₁-C₈)alkoxy, (C₁-C₈)alkanoyl, (C₁-C₈)alkylene, or heterocycle, is optionally partially unsaturated;
each R^{a}, R^{b} and R^{c} is independently hydrogen, (C₁-C₁₂)alkyl, (C₁-C₈)alkoxy, (C₁-C₈)alkoxy-(C₁-C₁₂)alkylene, (C₃-C₈)cycloalkyl, (C₃-C₈)cycloalkyl-(C₁-C₁₂)alkylene, (C₁-C₈)alkylthio, amino acid, aryl, aryl(C₁-C₈)alkylene, heterocycle, heterocycle-(C₁-C₈)alkylene, heteroaryl, or heteroaryl(C₁-C₈)alkylene;
alternatively R^{b} and R^{c}, together with the nitrogen to which they are attached, form a pyrrolidino, piperidino, morpholino, or thiomorpholino ring;
wherein any of the alkyl, cycloalkyl, heterocycle, aryl, or heteroaryl groups of R^{a}, R^{b} and R^{c} is optionally substituted on carbon with 1 or 2 substituents selected from the group consisting of halo, —(CH₂)ₐOR^{e}, —(CH₂)ₐSR^{e}, (C₁-C₈)alkyl, (CH₂)ₐCN, (CH₂)ₐNO₂, trifluoromethyl, trifluoromethoxy, —(CH₂)ₐCO₂R³, (CH₂)ₐNR^{e}R^{e}, and (CH₂)ₐC(O)NR^{e}R^{e};
R^{d} is hydrogen or (C₁-C₆)alkyl;
R^{e} is independently selected from H and (C₁-C₆)alkyl;
a is 0, 1, or 2;
i is 1 or 2
m is 0 to 8; and
p is 0 to 2;
provided that m is at least 1 when Z is NR⁴R⁵; or
a pharmaceutically acceptable salt thereof.

11. The method of claim 10, wherein the A_{2A} adenosine receptor agonist is a compound selected from the compounds of the following table or a stereoisomer or pharmaceutically acceptable salt thereof:

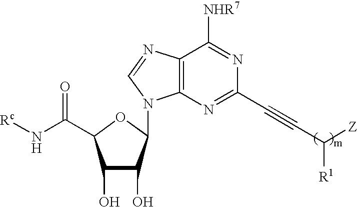

| Compound | | | |
|---|---|---|---|
| # | R^{c} | R⁷ | —(R¹)ₘ—Z |
| 1. | Et | H | 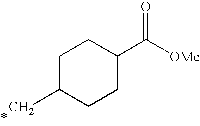 |
| 2. | Et | H | 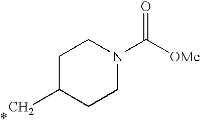 |
| 3. | cPr | H | 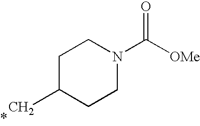 |
| 4. | Et | H | 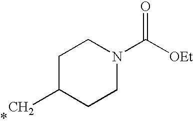 |
| 5. | cPr | H | 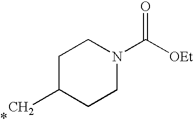 |
| 6. | Et | H | 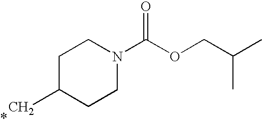 |
| 7. | cPr | H | 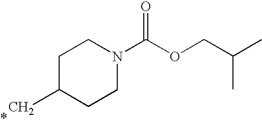 |
| 8. | Et | H | 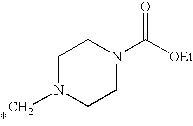 |
| 9. | Et | H | 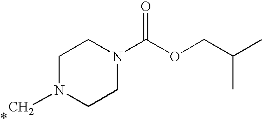 |
| 10. | Et | H | 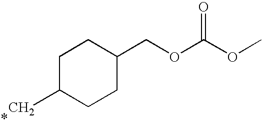 |
| 11. | Et | H | 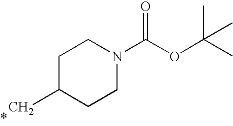 |
| 12. | cPr | H | 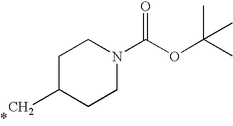 |
| 13. | Et | H | 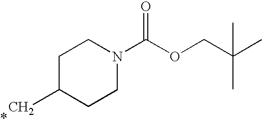 |
| 14. | cPr | H | 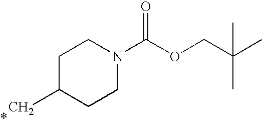 |
| 15. | Et | H | 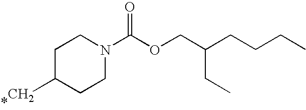 |
| 16. | cPr | H | 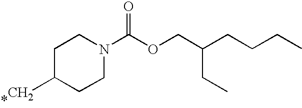 |
| 17. | cPr | H | 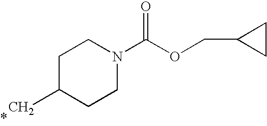 |
| 18. | Et | H | 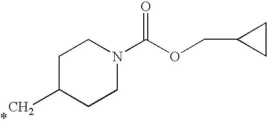 |
| 19. | cPr | H | 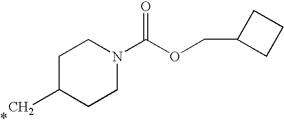 |
| 20. | Et | H | 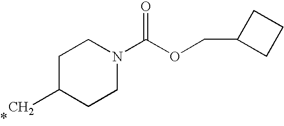 |
| 21. | cPr | H | 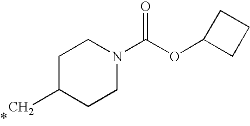 |
| 22. | Et | H | 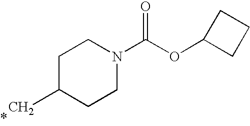 |
| 23. | Et | H | 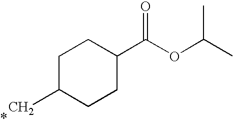 |
| 24. | cPr | H | 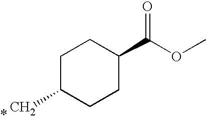 |
| 25. | Et | H | 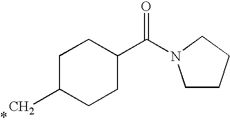 |
| 26. | Et | H | 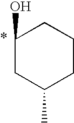 |
| 27. | Et | H | 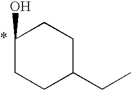 |
| 28. | Et | H | 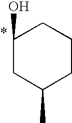 |
| 29. | Et | H | 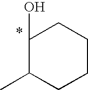 |
| 30. | Et | H | 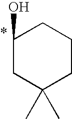 |
| 31. | cPr | H | 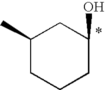 |
| 32. | Et | H | 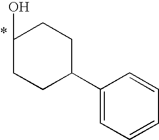 |
| 33. | Et | H | 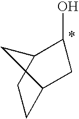 |
| 34. | cPr | H | 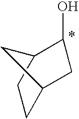 |
| 35. | cPr | H | 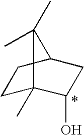 |
| 36. | Et | H | 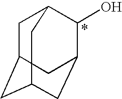 |
| 37. | cPr | H | 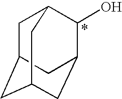 |
| 38. | Et | H | 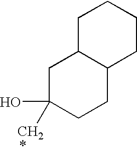 |
| 39. | cPr | H | 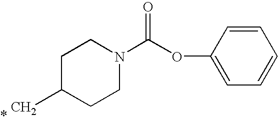 |
| 40. | Et | H | 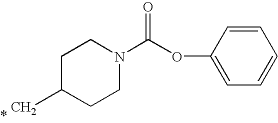 |
| 41. | cPr | H | 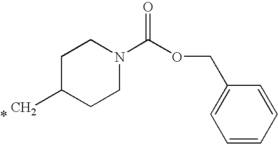 |
| 42. | Et | H | 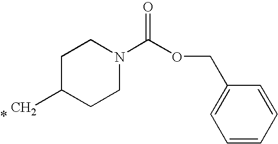 |

wherein * signifies the point of attachment.

12. The method of claim 1, wherein the A_{2A} adenosine receptor agonist is a compound of formula II or a stereoisomer or pharmaceutically acceptable salt thereof: 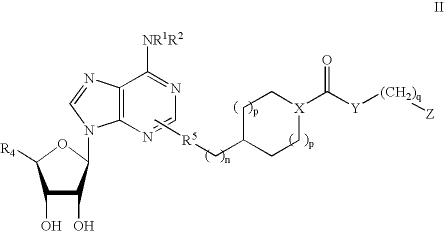
wherein:
R¹ and R² independently are selected from the group consisting of H, (C₁-C₈)alkyl, (C₃-C₈)cycloalkyl, (C₃-C₈)cycloalkyl(C₁-C₈)alkylene, aryl, aryl(C₁-C₈)alkylene, heteroaryl, heteroaryl(C₁-C₈)alkylene-, diaryl(C₁-C₈)alkylene, and diheteroaryl(C₁-C₈)alkylene, wherein the aryl and heteroaryl rings are optionally substituted with 1-4 groups independently selected from fluoro, chloro, iodo, bromo, methyl, trifluoromethyl, and methoxy;
each R independently is selected from the group consisting of H, C₁-C₄ alkyl, cyclopropyl, cyclobutyl, and (CH₂)ₐcyclopropyl;
X is CH or N, provided that when X is CH then Z cannot be substituted with halogen, C₁-C₆ alkyl, hydroxyl, amino, or mono- or di-(C₁-C₆-alkyl)amino;
Y is selected from the group consisting of O, NR¹, —(OCH₂CH₂O)ₘCH₂—, and —(NR¹CH₂CH₂O)ₘCH₂—, provided that when Y is O or NR¹, then at least one substituent is present on Z;
Z is selected from the group consisting of 5-membered heteroaryl, 6-membered aryl, 6-membered heteroaryl, carbocyclic biaryl, and heterocyclic biaryl, wherein the point of attachment of Y to Z is a carbon atom on Z, wherein Z is substituted with 0-4 groups independently selected from the group consisting of F, Cl, Br, I, (C₁-C₄)alkyl, —(CH₂)ₐOR³, (CH₂)ₐNR³R³, —NHOH, —NR³NR³R³, nitro, —(CH₂)ₐCN, —(CH₂)ₐCO₂R³, —(CH₂)ₐCONR³R³, trifluoromethyl, and trifluoromethoxy;
alternatively, Y and Z together form an indolyl, indolinyl, isoindolinyl, tetrahydroisoquinolinyl, or tetrahydroquinolinyl moiety wherein the point of attachment is via the ring nitrogen and wherein said indolyl, indolinyl, isoindolinyl, tetrahydroisoquinolinyl, or tetrahydroquinolinyl moiety, which is substituted with 0-4 groups independently selected from the group consisting of F, Cl, Br, I, C₁-C₄ alkyl, —(CH₂)ₐOR³, —(Cl₂)ₐNR³R³, —NHOH, —NR³NR³R³, NO₂, —(CH₂)ₐCN, —(CH₂)ₐCO₂R³, —(CH₂)ₐCONR³R³, CF₃, and OCF₃;
R³ is independently selected from the group consisting of H, (C₁-C₆)alkyl, cycloalkyl, aryl, and heteroaryl;
R⁴ is selected from the group consisting of CH₂OR, C(O)NRR, and CO₂R;
R⁵ is selected from the group consisting of CH₂CH₂, CH═CH, and C≡C;
a is selected from 0, 1, and 2;
m is selected from 1, 2, and 3;
n is selected from 0, 1, and 2;
each p independently is selected from 0, 1, and 2; and,
q is selected from 0, 1, and 2.

13. The method of claim 12, wherein the A_{2A} adenosine receptor agonist is a compound selected from the compounds of the following table or a stereoisomer or pharmaceutically acceptable salt thereof:
(i) 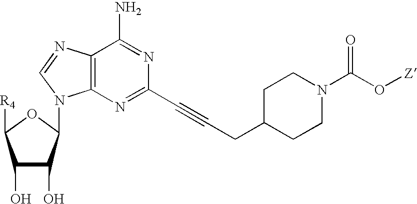
(ii) 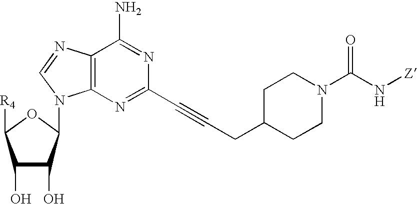
(iii)

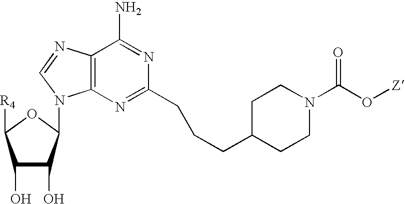

| Compound # | R⁴ | Z′ |
|---|---|---|
| 1 | C(O)NCyclopropyl | 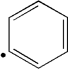 |
| 2 | C(O)NCyclopropyl | 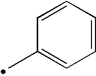 |
| 3 | C(O)NCyclopropyl | 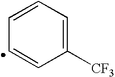 |
| 4 | CH₂OH | 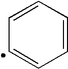 |
| 5 | C(O)NCyclopropyl | 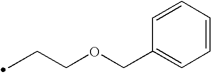 |
| 6 | CH₂OH | 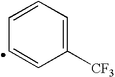 |
| 7 | CH₂OH | 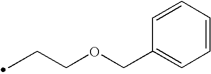 |
| 8 | C(O)NCyclopropyl | 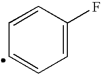 |
| 9 | C(O)NCyclopropyl | 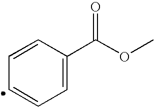 |
| 10 | C(O)NCyclopropyl | 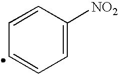 |
| 11 | CH₂OH | 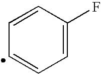 |
| 12 | CH₂OH | 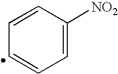 |
| 13 | CH₂OH | 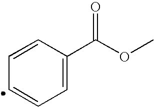 |
| 14 | C(O)NCyclopropyl | 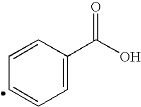 |
| 15 | C(O)NEthyl | 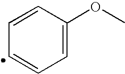 |
| 16 | C(O)NEthyl | 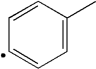 |
| 17 | C(O)NCyclopropyl | 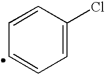 |
| 18 | C(O)NCyclopropyl | 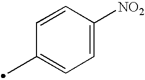 |
| 19 | C(O)NEthyl | 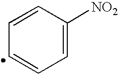 |
| 20 | C(O)NCyclopropyl | 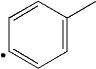 |
| 21 | C(O)NCyclopropyl | 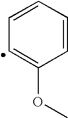 |
| 22 | C(O)NCyclopropyl | 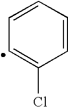 |
| 23 | C(O)NCyclopropyl | 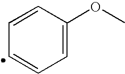 |
| 24 | C(O)NEthyl | 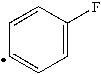 |
| 25 | C(O)NEthyl | 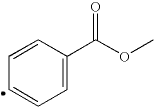 |
| 26 | C(O)NEthyl | 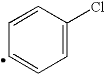 |
| 27 | CH₂OH | 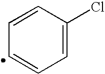 |
| 28 | CH₂OH | 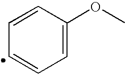 |
| 29 | CH₂OH | 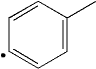 |
| 30 | CH₂OH | 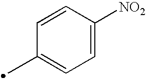 |
| 31 | C(O)NEthyl | 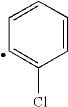 |
| 32 | C(O)NEthyl | 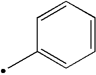 |
| 33 | C(O)NEthyl | 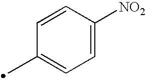 |
| 34 | C(O)NEthyl | 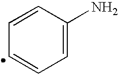 |
| 35 | CH₂OH | 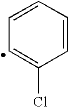 |
| 36 | CH₂OH | 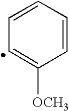 |
| 37(iii) | C(O)NEthyl | 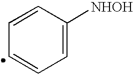 |
| 38(iii) | C(O)NCyclopropyl | 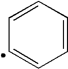 |
| 39(iii) | C(O)NCyclopropyl | 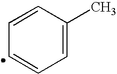 |
| 40(iii) | C(O)NCyclopropyl | 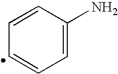 |
| 41(iii) | C(O)NCyclopropyl | 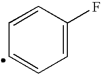 |
| 42 | C(O)NCyclopropyl | 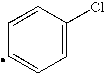 |
| 43(ii) | C(O)NCyclopropyl | 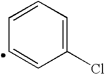 |
| 44(ii) | CH₂OH | 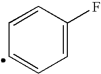 |
| 45(ii) | CH₂OH | 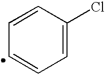 |
| 46(ii) | CH₂OH | 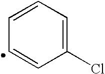 |
| 47(ii) | C(O)NCyclopropyl | 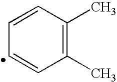 |
| 48(ii) | C(O)NCyclopropyl | 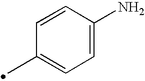 |
| 49 | C(O)NEthyl | 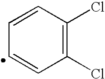 |
| 50 | C(O)NEthyl | 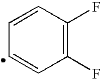 |
| 51 | C(O)NCyclopropyl | 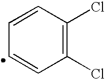 |
| 52 | C(O)NCyclopropyl | 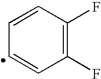 |
| 53 | CH₂OH | 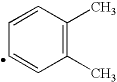 |
| 54 | CH₂OH | 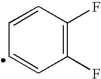 |
| 55 | CH₂OH | 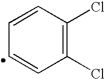 |
| 56 | C(O)NCyclopropyl | 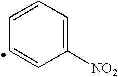 |
| 57 | C(O)NCyclopropyl | 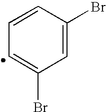 |

wherein • signifies the point of attachment.
wherein the compounds are formula (i), unless indicated

14. The method of claim 1, wherein the A_{2A} adenosine receptor agonist is a compound of formula (Ib)-(Id) or a pharmaceutically acceptable salt thereof:

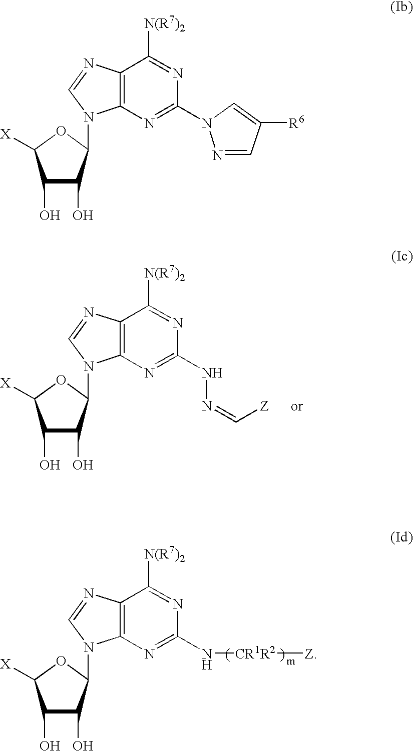

15. The method of claim 1, wherein the A_{2A} adenosine receptor agonist is selected from: 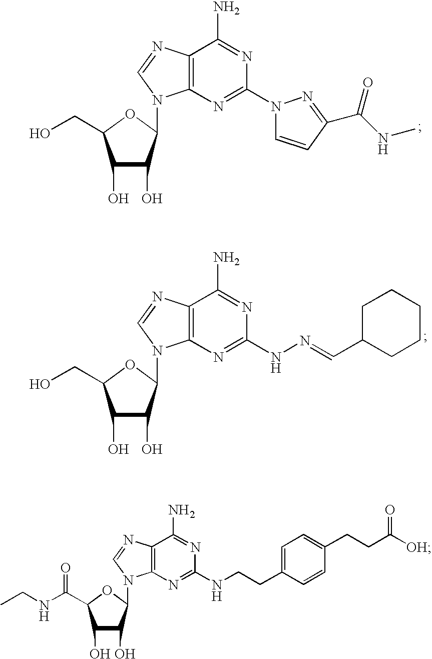
or a pharmaceutically acceptable salt thereof.

16. The method of claim 1, wherein the A_{2A} adenosine receptor agonist is a compound of the following formula or a pharmaceutically acceptable salt thereof:

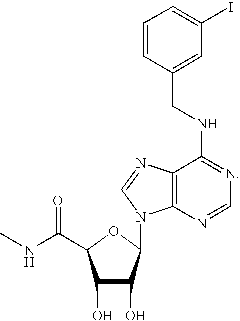

17. The method of claim 1, wherein the A_{2A} adenosine receptor agonist is a compound of the following formula or a pharmaceutically acceptable salt thereof: [chemical structure]
